(19) United States
(12) Patent Application Publication
(10) Pub. No.: US 2022/0195023 A1
(43) Pub. Date: Jun. 23, 2022

(54) ANTI-CSP ANTIBODY VARIANTS

(71) Applicant: Atreca, Inc., S. San Francisco, CA (US)

(72) Inventor: Shaun M. Lippow, San Carlos, CA (US)

(21) Appl. No.: 17/432,185

(22) PCT Filed: Feb. 19, 2020
(86) PCT No.: PCT/US2020/018745

§ 371 Date: Aug. 19, 2021

Related U.S. Application Data
(60) Provisional application No. 62/807,535, filed on Feb. 19, 2019.

(51) Int. Cl. C07K 16/20; A61P 33/06

(52) U.S. Cl. CPC C07K 16/205; A61K 2039/505; C07K 2317/92; A61P 33/06

(57) ABSTRACT

Provided herein are anti-circumsporozoite (CSP) antibodies, compositions comprising such antibodies, and methods of producing the antibodies. Additionally provided are methods of treating or preventing malaria using the anti-CSP antibodies.

### CROSS REFERENCE TO RELATED APPLICATION

This application claims priority to U.S. Provisional Application No. 62/807,535, filed on Feb. 19, 2019, the entire content of said provisional application is herein incorporated by reference for all purposes.

### BACKGROUND OF THE INVENTION

An anti-circumsporozoite (CSP) antibody that inhibits parasite development in vivo was obtained from a donor enrolled in a Phase 2a study evaluating efficacy of the Plasmodium falciparum circumsporozoite (CSP)-based malaria vaccine RTS,S. The RTS,S vaccine is a pseudo-viral particle vaccine that combines heptatits B surface antigen and the central repeat and C-terminal region of the CSP protein. The anti-CSP antibody (AB-000317) binds to CSP protein with high affinity, does not bind to hepatitis B protein, and originated in a donor who was protected from challenge with a controlled human malaria parasite infection (see, e.g., Oyen et al., Proc. Natl. Acad Sci. USA 114:E10438-E10445, 2017; Epub Nov. 14, 2017).

### BRIEF SUMMARY OF SOME ASPECTS OF THE INVENTION

The present disclosure is based, in part, upon the identification generation of variants of an antibody (referred to herein as AB-000317) obtained from a donor. In some embodiments, a variant antibody of the present disclosure exhibits superior developability and/or reduced immunogenicity compared to the parent antibody AB-000317. In some embodiments, the variant antibody also exhibits comparable or improved binding and/or in vivo activity as compared to AB-000317.

Thus, in one embodiment provided herein is an anti-circumsporozoite (CSP) antibody comprising a heavy chain variable (V_{H}) region and a light chain variable (V_{L}) region, wherein: (a) the V_{H} region comprises at least one substitution in a CDR1 sequence comprising ²⁶GFTFSTYAMH³⁵, a CDR2 sequence comprising ⁵⁰VISYHSTNKYYEDSVRG⁶⁶, and a CDR3 sequence comprising ⁹⁷ARDGYSSSFFDF¹⁰⁸ as numbered with reference to SEQ ID NO:1; wherein the at least one substitution is selected from the group consisting of D at position 30; S, D, or N at position 31; S at position 33; N or Q at position 53; D at position 54, G or E at position 55; S, R, or N at position 56; Q at position 57; A at position 61; E or S at position 62; K at position 65; A at position 100; A, T, Q, or H at position 102; T at position 103, T at position 104, Y at position 105; and Y at position 108; and (b) the V_{L} region comprises: (i) a CDR1 sequence comprising ²⁴RASQSISRWLA³⁴, a CDR2 sequence comprising ⁵⁰GASVLES⁵⁶, and a CDR3 sequence comprising ⁸⁹QHYNSYFVT⁹⁷ as numbered with reference to SEQ ID NO:2; or (ii) at least one substitution in the CDR1 sequence, CDR2 sequence, or CDR3 sequence, wherein the at least one substitution is selected from the group consisting of D or N at position 28; D at position 30; S at position 31; K, Q, S, L, V, or A at position 50; S or N at position 53; D at position 56; Q at position 90, Y at position 92; H at position 94, S or Y at position 95; and W at position 96. In some embodiments, if position 53 of the V_{H} region is N, position 55 is E. In a further embodiment, provided herein is an anti-CSP antibody comprising a heavy chain variable (V_{H}) region and a light chain variable (V_{L}) region, wherein: (a) the V_{H} region comprises: (i) a CDR1 sequence comprising ²⁶GFTFSTYAMH³⁵, a CDR2 sequence comprising ⁵⁰VISYHSTNKYYEDSVRG⁶⁶, and a CDR3 sequence comprising ⁹⁷ARDGYSSSFFDF¹⁰⁸ as numbered with reference to SEQ ID NO:1; or (ii) at least one substitution in the CDR1 sequence, the CDR2 sequence, or the CDR3 sequence, wherein the at least one substitution is selected from the group consisting of D at position 30; S, D, or N at position 31; S at position 33; N or Q at position 53; D at position 54, G or E at position 55; S, R, or N at position 56; Q at position 57; A at position 61; E or S at position 62; K at position 65; A at position 100; A, T, Q, or H at position 102; T at position 103, T at position 104, Y at position 105; and Y at position 108; and (b) the V_{L} region comprises at least one substitution in a CDR1 sequence comprising ²⁴RASQSISRWLA³⁴, a CDR2 sequence comprising ⁵⁰GASVLES⁵⁶, and a CDR3 sequence comprising ⁸⁹QHYNSYFVT⁹⁷ as numbered with reference to SEQ ID NO:2; wherein the at least one substitution is selected from the group consisting of D or N at position 28; D at position 30; S at position 31; K, Q, S, L, V, or A at position 50; S or N at position 53; D at position 56; Q at position 90, Y at position 92; H at position 94, S or Y at position 95; and W at position 96. In some embodiments, if position 53 of the V_{H} region is N, position 55 is E. In some embodiments, an antibody as described herein above comprises a V_{H} region that comprises at least one of the following, as numbered with reference to SEQ ID NO:1: V at position 2, A or E at position 23, A at position 40, K at position 43, or E at position 46. In some embodiments, the V_{L} region comprises at least one of the following, as numbered with reference to SEQ ID NO:2: T at position 20; K at position 39; I at position 48; or A, T, or Y at position 49. In some embodiments, the V_{H} region has at least 70% identity to SEQ ID NO:1; and the V_{L} region has at least 70% identity to SEQ ID NO:2. In some embodiments, the V_{H} region has at least 80% identity to SEQ ID NO:1; and/or the V_{L} region has at least 80% identity to SEQ ID NO:2. In further embodiments, the V_{H} region has at least 90% identity to SEQ ID NO: 1; and/or the V_{L} region has at least 90% identity to SEQ ID NO:2. In some embodiments, the V_{H} region has at least 95% identity to SEQ ID NO:1; and/or the V_{L} region has at least 95% identity to SEQ ID NO:2.

In some embodiments, provided herein is an anti-CSP antibody comprising a heavy chain variable (V_{H}) region and a light chain variable (V_{L}) region, wherein the V_{H} region has at least 70% identity to SEQ ID NO:1; and comprises a CDR1 as set forth in Table 1 or the CDR1 of Table 1 in which 1, 2, or 3 amino acids are substituted, a CDR2 as set forth in Table 1 or the CDR2 of Table 1 in which 1, 2, 3, 4, 5, 6, 7, 8, or 9 amino acids are substituted, and a CDR3 as set forth in Table 1 or the CDR3 of Table 1 in which 1, 2, 3, 4, 5, 6, or 7 amino acids are substituted; and at least one of the following substitutions as determined with reference to SEQ ID NO:1: V at position 2, A or E at position 23, A at position 40, K at position 43, or E at position 46; and the V_{L} region (i) comprises the amino acid sequence of SEQ ID NO:2; or (ii) comprises a CDR1 as set forth in Table 1 or the CDR1 of Table 1 in which 1, 2, 3, or 4 amino acids are substituted, a CDR2 as set forth in Table 1 or the CDR2 of Table 1 in which 1, 2, or 3 amino acids are substituted, and a CDR3 as set forth in Table 1 or the CDR3 of Table 1 in which 1, 2, 3, 4, 5, or 6 amino acids are substituted; and at least one of the following substitutions as determined with reference to SEQ ID NO:2: T at position 20; K at position 39; I at position 48; or A, T, or Y at position 49. In a further embodiment, provided herein is an anti-CSP antibody comprising a heavy chain variable (V_{H}) region and a light chain variable (V_{L}) region, wherein: the V_{H} region (i) comprises the amino acid sequence of SEQ ID NO:1; or (ii) has at least 70% identity to SEQ ID NO 1: and comprises a CDR1 as set forth in Table 1 or the CDR1 of Table 1 in which 1, 2, or 3 amino acids are substituted, a CDR2 as set forth in Table 1 or the CDR2 of Table 1 in which 1, 2, 3, 4, 5, 6, 7, 8, or 9 amino acids are substituted, and a CDR3 as set forth in Table 1 or the CDR3 of Table 1 in which 1, 2, 3, 4, 5, 6, or 7 amino acids are substituted; and at least one of the following substitutions as determined with reference to SEQ ID NO:1: V at position 2, A or E at position 23, A at position 40, K at position 43, or E at position 46; and the V_{L} region comprises an amino acid sequence having at least 70% identity to SEQ ID NO:2 and comprises a CDR1 as set forth in Table 1 or the CDR1 of Table 1 in which 1, 2, 3, or 4 amino acids are substituted, a CDR2 as set forth in Table 1 or the CDR2 of Table 1 in which 1, 2, or 3 amino acids are substituted, and a CDR3 as set forth in Table 1 or the CDR3 of Table 1 in which 1, 2, 3, 4, 5, or 6 amino acids are substituted; and at least one of the following substitutions as determined with reference to SEQ ID NO:2: T at position 20; K at position 39; I at position 48; or A, T, or Y at position 49. In some embodiments, such V_{H} regions comprise an amino acid sequence having at least 80% identity to SEQ ID NO:1; and/or the V_{L} region comprises an amino acid sequence having at least 80% identity to SEQ ID NO:2. In some embodiments, the V_{H} region comprises an amino acid sequence having at least 90% identity to SEQ ID NO:1; and/or the V_{L} region comprises an amino acid sequence having at least 90% identity to SEQ ID NO:2.

In some embodiments, provided herein is an anti-CSP antibody comprising a V_{H} region and V_{L} region, wherein the V_{H} region has at least 90% identity to any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, or 93; and comprises at least one substitution as determined with reference to SEQ ID NO:1, wherein the at least one substitution is selected from the group consisting of V at position 2; A at position 23; S, D, or N at position 31; S at position 33; A at position 40; E at position 46; N or Q at position 53; D at position 54; E at position 55; R or N at position 56; Q at position 57; A at position 61; E at position 62; K at position 65; A at position 100; A, T, Q, or H at position 102; T at position 103; T at position 104; Y at position 105; and Y at position 108; and the V_{L} region has at least 90% identity to any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, or 94; and comprises at least one substitution as determined with reference to SEQ ID NO:2, wherein the at least one substitution is selected from the group consisting T at position 20; D at position 28; D at position 30, S at position 31; I at position 48; A or T at position 49; K, Q, S, L, V, or A at position 50; S or N at position 53, D at position 56; Y at position 92; and S or Y at position 95.

In some embodiments, such an anti-CSP antibody comprises the following sets of amino acids:

(i) A at position 23 and A at position 40 as determined with reference to SEQ ID NO:1; and T at position 20 as determined with reference to SEQ ID NO:2;
(ii) E at position 46 and A at position 61 as determined with reference to SEQ ID NO:1;
(iii) V at position 2, A at position 23, and A at position 40 as determined with reference to SEQ ID NO:1; and T at position 20 and I at position 48 as determined with reference to SEQ ID NO:2;
(iv) A at position 23, A at position 40, E at position 46, and A at position 61 as determined with reference to SEQ ID NO:1; and T at position 20 as determined with reference to SEQ ID NO:2;
(v) V at position 2; A at position 23, A at position 40, E at position 46, and A at position 61 as determined with reference to SEQ ID NO:1; and T at position 20 and I at position 48 as determined with reference to SEQ ID NO:2;
(vi) N at position 53 and E at position 55 as determined with reference to SEQ ID NO: 1; or
(vii) A at position 23 and A at position 40 as determined with reference to SEQ ID NO: 1; and T at position 20 and Y at position 95 as determined with reference to SEQ ID NO:2.
In some embodiments, the V_{H} region has at least 95% identity to any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95; and/or the V_{L} region has at least 95% identity to any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96. In some embodiments, the V_{H} region comprises the amino acid sequence of any one of SEQ ID NOS:3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95; and/or the V_{L} region comprises the amino acid sequence of any one of SEQ ID NOS:4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96.

In some embodiments, an anti-CSP antibody provided herein comprises:

a V_{H} region comprising amino acid sequence SEQ ID NO:3; and a V_{L} region comprising amino acid sequence SEQ ID NO:4;
a V_{H} region comprising amino acid sequence SEQ ID NO:5; and a V_{L} region comprising amino acid sequence SEQ ID NO:6;
a V_{H} region comprising amino acid sequence SEQ ID NO:7; and a V_{L} region comprising amino acid sequence SEQ ID NO:8;
a V_{H} region comprising amino acid sequence SEQ ID NO:9; and a V_{L} region comprising amino acid sequence SEQ ID NO:10;
a V_{H} region comprising amino acid sequence SEQ ID NO: 11; and a V_{L} region comprising amino acid sequence SEQ ID NO:12;
a V_{H} region comprising amino acid sequence SEQ ID NO: 13; and a V_{L} region comprising amino acid sequence SEQ ID NO:14;
a V_{H} region comprising amino acid sequence SEQ ID NO:15; and a V_{L} region comprising amino acid sequence SEQ ID NO:16;
a V_{H} region comprising amino acid sequence SEQ ID NO: 17; and a V_{L} region comprising amino acid sequence SEQ ID NO:18;
a V_{H} region comprising amino acid sequence SEQ ID NO: 19; and a V_{L} region comprising amino acid sequence SEQ ID NO:20;
a V_{H} region comprising amino acid sequence SEQ ID NO:21; and a V_{L} region comprising amino acid sequence SEQ ID NO:22;
a V_{H} region comprising amino acid sequence SEQ ID NO:23; and a V_{L} region comprising amino acid sequence SEQ ID NO:24;
a V_{H} region comprising amino acid sequence SEQ ID NO:25; and a V_{L} region comprising amino acid sequence SEQ ID NO:26;
a V_{H} region comprising amino acid sequence SEQ ID NO:27 and a V_{L} region comprising amino acid sequence SEQ ID NO:28;
a V_{H} region comprising amino acid sequence SEQ ID NO:29 and a V_{L} region comprising amino acid sequence SEQ ID NO:30;
a V_{H} region comprising amino acid sequence SEQ ID NO:31 and a V_{L} region comprising amino acid sequence SEQ ID NO:32;
a V_{H} region comprising amino acid sequence SEQ ID NO:33 and a V_{L} region comprising amino acid sequence SEQ ID NO:34;
a V_{H} region comprising amino acid sequence SEQ ID NO:35 and a V_{L} region comprising amino acid sequence SEQ ID NO:36;
a V_{H} region comprising amino acid sequence SEQ ID NO:27 and a V_{L} region comprising amino acid sequence SEQ ID NO:38;
a V_{H} region comprising amino acid sequence SEQ ID NO:39 and a V_{L} region comprising amino acid sequence SEQ ID NO:40;
a V_{H} region comprising amino acid sequence SEQ ID NO:41 and a V_{L} region comprising amino acid sequence SEQ ID NO:42;
a V_{H} region comprising amino acid sequence SEQ ID NO:43 and a V_{L} region comprising amino acid sequence SEQ ID NO:44;
a V_{H} region comprising amino acid sequence SEQ ID NO:45 and a V_{L} region comprising amino acid sequence SEQ ID NO:46;
a V_{H} region comprising amino acid sequence SEQ ID NO:47 and a V_{L} region comprising amino acid sequence SEQ ID NO:48;
a V_{H} region comprising amino acid sequence SEQ ID NO:49 and a V_{L} region comprising amino acid sequence SEQ ID NO:50;
a V_{H} region comprising amino acid sequence SEQ ID NO:51 and a V_{L} region comprising amino acid sequence SEQ ID NO:52;
a V_{H} region comprising amino acid sequence SEQ ID NO:53 and a V_{L} region comprising amino acid sequence SEQ ID NO:54;
a V_{H} region comprising amino acid sequence SEQ ID NO:55 and a V_{L} region comprising amino acid sequence SEQ ID NO:56;
a V_{H} region comprising amino acid sequence SEQ ID NO:57 and a V_{L} region comprising amino acid sequence SEQ ID NO:58;
a V_{H} region comprising amino acid sequence SEQ ID NO:59 and a V_{L} region comprising amino acid sequence SEQ ID NO:60;
a V_{H} region comprising amino acid sequence SEQ ID NO:61 and a V_{L} region comprising amino acid sequence SEQ ID NO:62;
a V_{H} region comprising amino acid sequence SEQ ID NO:63 and a V_{L} region comprising amino acid sequence SEQ ID NO:64;
a V_{H} region comprising amino acid sequence SEQ ID NO:65 and a V_{L} region comprising amino acid sequence SEQ ID NO:66;
a V_{H} region comprising amino acid sequence SEQ ID NO:67 and a V_{L} region comprising amino acid sequence SEQ ID NO:68;
a V_{H} region comprising amino acid sequence SEQ ID NO:69 and a V_{L} region comprising amino acid sequence SEQ ID NO:50;
a V_{H} region comprising amino acid sequence SEQ ID NO:71 and a V_{L} region comprising amino acid sequence SEQ ID NO:72;
a V_{H} region comprising amino acid sequence SEQ ID NO:73 and a V_{L} region comprising amino acid sequence SEQ ID NO:74;
a V_{H} region comprising amino acid sequence SEQ ID NO:75 and a V_{L} region comprising amino acid sequence SEQ ID NO:76;
a V_{H} region comprising amino acid sequence SEQ ID NO:77 and a V_{L} region comprising amino acid sequence SEQ ID NO:78;
a V_{H} region comprising amino acid sequence SEQ ID NO:79 and a V_{L} region comprising amino acid sequence SEQ ID NO:80;
a V_{H} region comprising amino acid sequence SEQ ID NO:81 and a V_{L} region comprising amino acid sequence SEQ ID NO:82;
a V_{H} region comprising amino acid sequence SEQ ID NO:83 and a V_{L} region comprising amino acid sequence SEQ ID NO:84;
a V_{H} region comprising amino acid sequence SEQ ID NO:85 and a V_{L} region comprising amino acid sequence SEQ ID NO:86;
a V_{H} region comprising amino acid sequence SEQ ID NO:87 and a V_{L} region comprising amino acid sequence SEQ ID NO:88;
a V_{H} region comprising amino acid sequence SEQ ID NO:89 and a V_{L} region comprising amino acid sequence SEQ ID NO:90;
a V_{H} region comprising amino acid sequence SEQ ID NO:91 and a V_{L} region comprising amino acid sequence SEQ ID NO:92;
a V_{H} region comprising amino acid sequence SEQ ID NO:93 and a V_{L} region comprising amino acid sequence SEQ ID NO:94; or
a V_{H} region comprising amino acid sequence SEQ ID NO:95 and a V_{L} region comprising amino acid sequence SEQ ID NO:96.

In a further aspect, provided herein is an expression vector comprising a polynucleotide encoding the V_{H} region and/or the V_{L} region of the anti-CSP antibody of any one of the preceding paragraphs in this section. Also provided herein is a host cell comprising an expression vector encoding the V_{H} and/or V_{L} region of the anti-CSP antibody; and a host cell comprising a polynucleotide that encodes the V_{H} region and/or the V_{L} region of the anti-CSP antibody. In a further aspect, provided herein is a method of producing any of the anti-CSP antibodies described above, and the method comprises culturing the host cell described above in a culture medium.

In a further aspect, provided herein is a method of treating or preventing malaria, the method comprising administering the anti-CSP antibody as described herein to a patient that has malaria or is at risk of contracting malaria.

In further aspect, provided herein is the use of the an anti-CSP antibody of an antibody as described herein for treating or preventing malaria; and the use of the antibody for preparation of a medicament for treating or preventing malaria.

### BRIEF DESCRIPTION OF THE DRAWINGS

FIG. 1 shows an alignment of AB-000317 heavy and light chain variable region sequences with germline sequences. CDR sequences are shaded.

FIGS. 2A and 2B show K_{D} values for 47 variants of AB-000317 from biolayer interferometry (BLI; FIG. 2A) and surface plasmon resonance (SPR; FIG. 2B) assays for binding various targets including CSP and peptides derived from it. For each plot, K_{D} (nM) is plotted on the y-axis and the target against which antibody binding was measured is specified on the x-axis. Antibodies with stronger binding have lower K_{D} values while antibodies with weaker binding have higher K_{D} values. Each circle indicates the binding strength for one antibody in the specified peptide-assay combination. The longer, solid bar in each graph represents the average binding for parent antibody AB-000317, and the shorter, dashed bar identifies average AB-000317 variant binding. The percentage of antibodies that bound to each peptide target is shown above the data points for that target.

FIG. 3A-3E show K_{D} measurements for 47 AB-000317 variant and parent antibodies in biolayer interferometry (BLI) or surface plasmon resonance (SPR) assays. The antibodies are divided by variant category as indicated along the x-axis, as described in Table 12A and 12B. The variant in the “Combo” category, i.e., AB-007074, was designed to have a combination of selected modifications from other categories. Regions of each plot shaded in grey represent a 3-fold difference as compared to the average AB-000317 binding. The data points within the shaded region represent antibodies having target binding comparable to the parent antibody, AB-000317. The data points below the shaded region represent antibodies having increased binding to the target relative to the parent antibody.

FIG. 4 shows results of testing anti-CSP antibody variants in a liver burden malaria mouse model. Bioluminescence (photons/sec) generated from the fluorescent sporozoites is measured on the y-axis. The circles indicate the total amount of bioluminescence measured in a single mouse and by extension, the sporozoite liver burden. AB-001245 is a non-malaria-specific antibody that was used as a negative control.

FIG. 5 shows Kabat, Chothia, and IMGT numbering of AB-000317.

### DETAILED DESCRIPTION OF THE DISCLOSURE

### Terminology

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of antibody or fragment thereof that exhibits the desired biological activity, e.g., binding the specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

As used herein, the terms, “anti-CSP antibody” and “CSP antibody” are used synonymously to refer to an antibody that binds to Plasmodium falciparium cirucumsporozoite (CSP) antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

As used herein, “V-region” refers to an antibody variable region domain comprising the segments of Framework 1, CDR1, Framework 2, CDR2, Framework 3, CDR3, and Framework 4. The heavy chain V-region, V_{H}, is a consequence of rearrangement of a V-gene (HV), a D-gene (HD), and a J-gene (HJ), in what is known as V(D)J recombination during B-cel differentiation. The light chain V-region, VL, is a consequence of rearrangement of a V-gene (LV) and a J-gene (LJ).

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the particular CDR is located. Thus, a V_{H} CDR3 (HCDR3) is located in the variable domain of the heavy chain of the antibody in which it is found, whereas a V_{L} CDR3 (LCDR3) is the CDR3 from the variable domain of the light chain of the antibody in which it is found. The term “CDR” is used interchangeably with “HVR” when referring to CDR sequences.

The amino acid sequences of the CDRs and framework regions can be determined using various well known definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, Md. (1991)). Chothia CDRs are determined as defined by Chothia (see, e.g., Chothia and Lesk J. Mol. Biol. 196:901-917 (1987)).

An “Fc region” refers to the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modifications that modulate effector function. Fc regions also include variants that don't do not result in alterations to biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibody (see, e.g., Angal, et al., Mol Immunol 30:105-108, 1993).

The term “equilibrium dissociation constant” abbreviated (K_{D}), refers to the dissociation rate constant (k_{d}, time⁻¹) divided by the association rate constant (kₐ, time⁻¹ M⁻¹). Equilibrium dissociation constants can be measured using any method. Thus, in some embodiments antibodies of the present disclosure have a K_{D} of less than about 50 nM, typically less than about 25 nM, or less than 10 nM, e.g., less than about 5 nM or than about 1 nM and often less than about 10 nM as determined by surface plasmon resonance analysis using a biosensor system such as a Biacore® system performed at 37° C. In some embodiments, an antibody of the present disclosure has a K_{D} of less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a bivalent antibody. In the context of the present invention, an “improved” K_{D}refers to a lower K_{D}. In some embodiments, an antibody of the present disclosure has a K_{D} of less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹²M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a monovalent antibody, such as a monovalent Fab. In some embodiments, an anti-CSP antibody of the present disclosure has K_{D} less than 100 pM, e.g., or less than 75 pM, e.g., in the range of 1 to 100 pM, when measured by surface plasmon resonance analysis using a biosensor system such as a Biacore® system performed at 37° C. In some embodiments, an anti-CSP antibody of the present disclosure has K_{D} of greater than 100 pM, e.g., in the range of 100-1000 pM or 500-1000 pM when measured by surface plasmon resonance analysis using a biosensor system such as a Biacore® system performed at 37° C.

The term “monovalent molecule” as used herein refers to a molecule that has one antigen-binding site, e.g., a Fab or scFv.

The term “bivalent molecule” as used herein refers to a molecule that has two antigen-binding sites. In some embodiments, a bivalent molecule of the present invention is a bivalent antibody or a bivalent fragment thereof. In some embodiments, a bivalent molecule of the present invention is a bivalent antibody. In some embodiments, a bivalent molecule of the present invention is an IgG. In general monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” as used herein refer to the binding of both antigen-binding sites of a bivalent molecule to its antigen. Preferably both antigen-binding sites of a bivalent molecule share the same antigen specificity.

The term “valency” as used herein refers to the number of different binding sites of an antibody for an antigen. A monovalent antibody comprises one binding site for an antigen. A bivalent antibody (e.g., a bivalent IgG antibody) comprises two binding sites for the same antigen.

The term “affinity” as used herein refers to either the single or combined strength of one or both arms of an antibody (e.g., an IgG antibody) binding to either a simple or complex antigen expressing one or more epitopes. As defined here, the term “affinity” does not imply a specific number of valencies between the two binding partners.

The phrase “specifically (or selectively) binds” to an antigen or target or “specifically (or selectively) immunoreactive with,” when referring to a protein or peptide, refers to a binding reaction whereby the antibody binds to the antigen or target of interest. In the context of this invention, the antibody selectively binds to Plasmodium falciparum: CSP protein in the (NPNA)₃ region.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a V_{H} region polypeptide “corresponds to” an amino acid in the V_{H} region of SEQ ID NO:1 when the residue aligns with the amino acid in SEQ ID NO:1 when optimally aligned to SEQ ID NO:1. The polypeptide that is aligned to the reference sequence need not be the same length as the reference sequence.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence.

The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cells and includes the primary transformed cell and progeny derived therefrom without regard to the number of passages.

A polypeptide “variant,” as the term is used herein, is a polypeptide that typically differs from a polypeptide specifically disclosed herein in one or more substitutions, deletions, additions and/or insertions. In the present invention, a “variant” with reference to the sequences described in the “Anti-CSP Antibody Variants” section refers to a engineered sequence, rather than a naturally occurring sequence.

The term “comparable,” in the context of describing the strength of binding of two antibodies to the same target, refers to two dissociation constant (KD) values calculated from two binding reactions that are within three (3) fold from each other. In other words, the ratio between the first K_{D} (the K_{D} of the binding reaction between the first antibody and the target) and the second K_{D} (the K_{D} of the binding reaction between the second antibody and the target) is within the range of 1:3 or 3:1, endpoints exclusive. A lower K_{D} value denotes stronger binding. For example, an antibody variant that has stronger binding as compared to AB-000317 binds to the target with a K_{D} that is at least ⅓ of the K_{D} measured against the same target for AB-000317.

### Anti-CSP Antibody Variants

Provided herein are anti-CSP antibody variants of antibody AB-000317 that was obtained from a vaccinated human who was subsequently protected from a controlled malaria infection. Such variants exhibit protective effects in vivo, e.g., as shown by a reduction in parasite number in a mouse model of malaria infection. In some embodiments, an anti-CSP antibody of the present disclosure comprises modifications compared to AB-000317 that provide improved pharmacokinetic properties, increased serum stability, stronger binding, and/or in vivo protective effects compared to AB-000317. In some embodiments, a variant antibody as described herein exhibits reduced immunogenicity and/or increased manufacturability as compared to AB-000317. In some embodiments, a variant anti-CSP antibody having at least one modification, e.g., substitution, relative to the native AB-000317 variable heavy chain or light chain sequence as described herein has improved developability, e.g., decreased heterogeneity, increased yield, increased stability, improved net charges to improve pharmacokinetics, and or/reduced immunogenicity. In some embodiments, a V_{H} region or a V_{L} region of such an antibody has at least two, three, four, five, or six, or more modifications, e.g., substitutions, as described herein. In some embodiments, a variant anti-CSP antibody of the invention has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g. substitutions, including both variable regions, compared to AB-000317.

The variable region sequences of AB-000317 are provided in Table 1:

TABLE 1
AB-000317 variable region sequences

| Antibody | | |
|---|---|---|
| Designation | V_{H} amino acid sequence | V_{L} amino acid sequence |
| AB-000317 | QMQLVESGGGVVQPGRSLRLSCTAS | DIQMTQSPSTLSASVGDRVAIT |
| | GFTFSTYAMHWVRQSPGQGLQWVA | CRASQSISRWLAWYQQQPGKA |
| | VISYHSTNKYYEDSVRGRFTISRDNS | PKLLMSGASVLESGVPSRFSGS |
| | KNTLYLQMNSLRAEDTAVYYCARD | GSGTEFTLTISSLQPDDFATYYC |
| | GYSSSFFDFWGQGTLVTVSS (SEQ | QHYNSYFVTFGQGTKVEIK |
| | ID NO: 1) | (SEQ ID NO: 2) |

The heavy and light chain CDRs of AB-000317 as shown in Table 2 are defined by IMGT and Kabat.

TABLE 2
AB-000317 CDR sequences

| | CDR1 | CDR2 | CDR3 |
|---|---|---|---|
| 317 V_{H} | GFTFSTYAMH | VISYHSTNKYYEDSVRG | ARDGYSSSFFDF |
| region | (SEQ ID NO: 97) | (SEQ ID NO: 98) | (SEQ ID NO: 99) |
| 317 V_{L} | RASQSISRWLA | GASVLES | QHYNSYFVT |
| region | (SEQ ID NO: 100) | (SEQ ID NO: 101) | (SEQ ID NO: 102) |

Position 119 of SEQ ID NO:1 and position 107 of SEQ ID NO:2 are considered to be the last amino acids of the V_{H} and V_{L} regions, respectively, according to EU index numbering. In a human IgG format (e.g., IgG1, IgG2, IgG3, or IgG4), the subsequent residue is termed the “junction codon”, and is natively encoded by the junction of the final 3′ base of the variable region gene (HJ or LJ) with the first two 5′ bases of the constant region gene (heavy or light), and exhibits amino acid variation due to variation in the final 3′ base of HJ and U. The human heavy chain junction codon can natively be Ala, Ser, Pro, or Thr, and is usually an Ala. The human kappa chain junction codon can natively be Arg or Gly, and is usually an Arg. The human lambda chain junction codon can natively be Gly, Ser, Arg, or Cys, and is usually a Ser or Gly.

The heavy chain CDRs encompass amino acid residues from amino acid residues 26-35 (HCDR1), 50-66 (HCDR2) and 97-108 (HCDR3). The light chain CDRs encompass amino acid residues from amino acid residues 24-34 (LCDR1), 50-56 (LCDR2), and 89-97 (LCDR3). The numbering of the residues corresponds to the positions in the V_{H} and V_{L} sequences in Table 2. The V_{H} CDRs, as listed in Table 2 are defined as follows: HCDR1 is defined by the union of Kabat and IMGT (i.e., each residue is defined as a residue in the HCDR1 if it is determined to be such according to either Kabat or IMGT); HCDR2 is defined by Kabat; and the HCDR3 is defined by IMGT. The V_{L} CDRs as listed in Table 2 are defined by Kabat. As known in the art, numbering and placement of the CDRs can differ depending on the numbering system employed. It is understood that disclosure of a variable heavy and/or variable light sequence includes the disclosure of the associated CDRs, regardless of the numbering system employed.

Using AB-000317 as a reference sequence, FIGS. 5A and 5B shows the numbering of the residues in the AB-000317 V_{H} and V_{L} sequences using IMGT, Kabat, and Chothia numbering systems.

The CDRs as defined using the IMGT numbering system are:
HCDR1: 27-38 (excluding positions 31-34)
HCDR2: 56-65 (excluding positions 60-61)
HCDR3: 105-117 (excluding position 111)
LCDR1: 27-38 (excluding positions 30-35)
LCDR2: 56-65 (excluding positions 58-64)
LCDR3: 105-117 (excluding positions 110-113).

Accordingly, the corresponding IMGT CDRs for AB-000317 are:

HCDR1:
GFTFSTYA
HCDR2:
ISYHSTNK
HCDR3:
ARDGYSSSFFDF
LCDR1:
QSISRW
LCDR2:
GAS
LCDR3:
QHYNSYFVT.

The CDRs as defined using the Kabat numbering system are:

### HCDR1: 31-35

HCDR2: 50-65 (including insertion of 52A)
HCDR3: 95-102 (including insertions of 100A and 100B)

### LCDR1: 24-34

### LCDR2: 50-56

### LCDR3: 89-97

Accordingly, the corresponding KABAT CDRs for AB-000317 are:

HCDR1:
TYAMH
HCDR2:
VISYHSTNKYYEDSVRG
HCDR3:
DGYSSSFFDF
LCDR1:
RASQSISRWLA
LCDR2:
GASVLES
LCDR3:
QHYNSYFVT

The CDRs defined using the Chothia numbering system are:

### HCDR1: 26-32

HCDR2: 52-56 (including insertion of 52A)
HCDR3: 95-102 (including insertions of 100A and 100B)

### LCDR1: 24-34

### LCDR2: 50-56

### LCDR3: 89-97.

Accordingly, the corresponding Chothia CDRs for AB-000317 are:

HCDR1:
GFTFSTY
HCDR2:
SYHSTN
HCDR3:
DGYSSSFFDF
LCDR1:
RASQSISRWLA
LCDR2:
GASVLES
LCDR3:
QHYNSYFVT

### V_{H} Region

In some embodiments, an anti-CSP antibody of the present invention has one, two, or three CDRs of a V_{H} sequence in Table 1. In some embodiments, an anti-CSP antibody has at least one mutation and no more than 10, 20, 30, 40 or 50 mutations in the V_{H} amino acid sequences compared to the V_{H} sequence of AB-000317. In some embodiments, the V_{H} region comprises 1 or 2 substitutions relative to the CDR1, CDR2 or CDR3 sequence shown in Table 2. In some embodiments, the V_{H} region comprises a CDR1 having 1, 2, or 3 substitutions in relative to the CDR1 sequence shown in Table 2. In some embodiments, the V_{H} region comprises a CDR 2 that has 1, 2, 3, or 4 substitutions relative to the CDR2 sequence shown in Table 2. In some embodiments, the V_{H} region has 5, 6, 7, 8, or 9 substitutions relative to the CDR2 sequence shown in Table 2. In some embodiments, the V_{H} region comprises a CDR3 that has 1, 2, or 3 substitutions relative to the CDR3 sequence shown in Table 2. In some embodiments, the V_{H} region has 4, 5, or 6 substitutions relative to the CDR3 sequence shown in Table 2. In some embodiments, one or more of the substitutions is a conservative substitution.

In some embodiments, an anti-CSP antibody of the present invention has a V_{H} that comprises a CDR1 sequence as shown in Table 2 in which one of positions 30, 31, and 33, as determined with reference to SEQ ID NO:1 are substituted; or in which two or all three positions are substituted. In some embodiments, the CDR1 sequence comprises one of the following substitutions, relative to the AB-000317 CDR1 sequence shown in Table 2: D at position 30; S, D, or N at position 31; or S at position 33. In some embodiments, the CDR1 sequence comprises two of the following substitutions, relative to the AB-000317 CDR1 sequence shown in Table 2: D at position 30; S, D, or N at position 31; or S at position 33. In some embodiments, the CDR2 sequence comprises the following substitutions, relative to the AB-000317 CDR1 sequence shown in Table 2: D at position 30; S, D, or N at position 31; or S at position 33

In some embodiments, the V_{H} region comprises the CDR2 sequence shown Table 2 in which one or more of positions 53, 54, 55, 56, 57, 61, 62, and 65, as numbered with reference to SEQ ID NO:1, is substituted. In some embodiment, the substitution is selected from the group consisting of N or Q at position 53; D at position 54, G or E at position 55; S, R, or N at position 56; Q at position 57; A at position 61; E or S at position 62; and K at position 65. In some embodiments, the CDR2 comprises a substitution at position 53, 54, 55, 56, 57, 61, 62, and 65 as designated in the preceding sentence and 1, 2, 3, or 4 additional substitutions in the CDR2 sequence. In some embodiments, the CDR2 comprises substitutions at two or three of positions 53, 54, 55, 56, 57, 61, 62, and 65, wherein the substitutions are selected from the group consisting of N or Q at position 53; D at position 54, G or E at position 55; S, R, or N at position 56; Q at position 57; A at position 61; E or S at position 62; and K at position 65. In some embodiments, the CDR2 comprises substitutions at four, five or six of positions 53, 54, 55, 56, 57, 61, 62, and 65, wherein the substitutions are selected from the group consisting of N or Q at position 53; D at position 54, G or E at position 55; S, R, or N at position 56; Q at position 57; A at position 61; E or S at position 62; and K at position 65. In some embodiments, the CDR2 comprises substitutions at seven or eight of positions 53, 54, 55, 56, 57, 61, 62, and 65, wherein the substitutions are selected N or Q at position 53; D at position 54, G or E at position 55; S, R, or N at position 56; Q at position 57; A at position 61; E or S at position 62; and K at position 65. In some embodiments, the CDR2 has at least 70% identity, or at least 80% identity, to the CDR2 sequence set forth in Table 2 and comprises at least one substitution at position 53, 54, 55, 56, 57, 61, 62, 63, and 65 wherein the substitutions are selected from the group consisting of N or Q at position 53; D at position 54, G or E at position 55; S, R, or N at position 56; Q at position 57; A at position 61; E or S at position 62; and K at position 65.

In some embodiments, an anti-CSP antibody of the present invention has a V_{H} that comprises a CDR3 sequence as shown in Table 2 in which one or two, of positions 100, 102, 103, 104, 105, or 108, as numbered with reference to SEQ ID NO:1, are substituted; or in which three or four of positions 100, 102, 103, 104, 105, or 108 are substituted. In some embodiments, five or six of positions 100, 102, 103, 104, 105, or 108 are substituted. In some embodiments, the V_{H} region comprises the CDR3 sequence shown Table 2 in which one position 100, 102, 103, 104, 105, or 108, as numbered with reference to SEQ ID NO:1, is substituted and the substitution is selected from the group consisting A at position 100; A, T, Q, or H at position 102; T at position 103, T at position 104, Y at position 105; and Y at position 108. In some embodiments, the CDR3 comprises a substitution at position 100, 102, 103, 104, 105, or 108 as designated in the preceding sentence and 1, 2, 3, or 4 additional substitutions. In some embodiments, the CDR3 comprises substitutions at two or three of positions 100, 102, 103, 104, 105, or 108, wherein the substitutions are selected from the group consisting of A at position 100; A, T, Q, or H at position 102; T at position 103, T at position 104, Y at position 105; and Y at position 108. In some embodiments, the CDR3 comprises substitutions at four, five, or six of positions 100, 102, 103, 104, 105, or 108, wherein the substitutions are selected from the following: A at position 100; A, T, Q, or H at position 102; T at position 103, T at position 104, Y at position 105; and Y at position 108. In some embodiments, the CDR3 has at least 70% identity, or at least 80% identity, to the CDR3 sequence set forth in Table 2 and comprises at least one substitution at position 100, 102, 103, 104, 105, or 108; wherein the substitutions is selected from the group consisting of A at position 100; A, T, Q, or H at position 102; T at position 103, T at position 104, Y at position 105; and Y at position 108.

In some embodiments, an anti-CSP antibody of the present invention comprises a V_{H} region CDR1, CDR2, and/or a CDR3 as described in the preceding three paragraphs. In some embodiments one or two of the V_{H} CDR sequence comprise an AB-000317 sequence as shown in Table 2. In some embodiments, an anti-CSP antibody of the present invention comprises a V_{H} region CDR1, CDR2 and/or a CDR3 as described in the preceding two paragraphs and has at least 70% identity, at least 75% identity, at least 80% identity, or at least 85% identity, at least 90% identity, or at least 95% identity to SEQ ID NO:1 In some embodiments, the V_{H} region further comprises at least one of the following, as numbered with reference to SEQ ID NO:1: V at position 2, A or E at position 23, A at position 40, K at position 43, or E at position 46.

In some embodiments, an anti-CSP antibody comprises a CDR1, CDR2 and/or a CDR3 as described in the previous paragraphs in this section and comprises two, three, four, or five additional amino acid changes relative to SEQ ID NO:1, but no more than thirty, or no more than thirty-five, additional changes. In some embodiments, the antibody comprises at least six, seven, eight, nine or ten additional amino changes relative to SEQ ID NO:1, but no more than thirty, or thirty-five, additional changes.

In some embodiments, an anti-CSP antibody of the present invention has at least 70% identity, at least 75% identity, at least 80% identity, or at least 85% identity, at least 90% identity, or at least 95% identity to SEQ ID NO:1 and comprises one or more of the following: V at position 2, A or E at position 23; D at position 30; S, D, or N at position 31; S at position 33; A at position 40, K at position 43, E at position 46, N or Q at position 53; D at position 54, G or E at position 55; S, R, or N at position 56; Q at position 57; A at position 61; E or S at position 62; K at position 65; A at position 100; A, T, Q, or H at position 102; T at position 103, T at position 104, Y at position 105; and Y at position 108. In some embodiments, when position 53 is N, position 55 is E. In some embodiments, an anti-CSP antibody comprises a V_{H} region that comprises the residue, or the combination of residues, as designated in column 3 of Table 10 in the EXAMPLES section.

### V_{L} Region

In some embodiments, an anti-CSP antibody of the present invention has at least one, at least two, or three CDRs of a V_{L} sequence of the antibody AB-000317 shown in Table 1; and at least one mutation, e.g., a deletion, substitution, or addition, in the amino acid sequence of the V_{L} region of the antibody compared to the AB-000317 V_{L} sequence. In some embodiments, the CDR1 comprises one, two, or three substitutions compared to the CDR1 of Table 2. In some embodiments, the CDR2 one, two, or three substitutions relative to the CDR2 sequence of Table 2. In some embodiments, the CDR3 comprises one, two, three, four, five, or six substitutions relative to the CDR3 sequence of Table 2.

In some embodiments, an anti-CSP antibody of the present invention has a V_{L} that comprises a CDR1 sequence as shown in Table 2 in which one of positions 28, 30, or 31 is substituted. In some embodiments, the substitution is selected from the group consisting of D or N at position 28; D at position 30; and S at position 31. In some embodiments, the CDR1 comprises 1 or 2 additional substitutions, e.g., conservative substitutions, relative to the CDR1 sequence set forth in Table 2. In some embodiments, the CDR1 comprises two substitutions selected from the group consisting of D or N at position 28; D at position 30; and S at position 31. In some embodiments, the antibody comprises three substitutions selected from the group consisting of D or N at position 28; D at position 30; and S at position 31. In some embodiments, the antibody comprises the following substitutions: D or N at position 28; D at position 30; and S at position 31.

In some embodiments, an anti-CSP antibody of the present invention has a V_{L} that comprises a CDR2 sequence as shown in Table 2 in which one of positions 50, 53, and 56 are substituted. In some embodiments, the substitution is selected from the group consisting of K, Q, S, L, V, or A at position 50; S or N at position 53; and D at position 56. In some embodiments, the CDR2 comprises one or two additional substitutions, e.g., conservative substitutions, relative to the CDR1 sequence set forth in Table 2. In some embodiments, the CDR2 comprises two substitutions selected from the group consisting of K, Q, S, L, V, or A at position 50; S or N at position 53; and D at position 56. In some embodiments, the antibody comprises three substitutions as follows: K, Q, S, L, V, or A at position 50; S or N at position 53; and D at position 56.

In some embodiments, an anti-CSP antibody of the present invention has a V_{L} that comprises a CDR3 sequence as shown in Table 2 in which one of positions 90, 92, 94, 95, or 96 is substituted. In some embodiments, the substitution is selected from the group consisting of Q at position 90, Y at position 92; H at position 94, S or Y at position 95; and W at position 96. In some embodiments, the CDR3 comprises 1 or 2 additional substitutions, e.g., conservative substitutions, relative to the CDR3 sequence set forth in Table 2. In some embodiments, the CDR3 comprises two substitutions selected from the group consisting Q at position 90, Y at position 92; H at position 94, S or Y at position 95; and W at position 96. In some embodiments, the CDR3 comprises three substitutions selected from the group consisting of Q at position 90, Y at position 92; H at position 94, S or Y at position 95; and W at position 96. In some embodiments, the antibody comprise four substitutions selected from the group consisting of Q at position 90, Y at position 92; H at position 94, S or Y at position 95; and W at position 96. In some embodiments, the antibody comprises five substitutions selected form the group consisting of Q at position 90, Y at position 92; H at position 94, S or Y at position 95; and W at position 96.

In some embodiments, an anti-CSP antibody of the present invention comprises a V_{L} region CDR1, CDR2, and/or a CDR3 as described in the previous paragraphs. In some embodiments, one or two of the CDRs are the native sequence shown in Table 2. In some embodiments, the V_{L} region has at least 70% identity, at least 75% identity, at least 80% identity, or at least 85% identity, at least 90% identity, or at least 95% identity to SEQ ID NO:2. In some embodiments, an antibody having a substitution in a V_{L} CDR1, CDR2, and/or CDR3 further comprises at least one of the following, as numbered with reference to SEQ ID NO:2: T at position 20; K at position 39; I at position 48; or A, T, or Y at position 49.

In some embodiments, an anti-CSP antibody comprises a V_{L} region CDR1, CDR2 and/or a CDR3 as described in the previous paragraphs in this section and comprises two, three, four, or five additional amino acid changes relative to SEQ ID NO:2, but no more than thirty additional changes. In some embodiments, the antibody comprises at least six, seven, eight, nine or ten additional amino changes relative to SEQ ID NO:2, but no more than, twenty five, or no more than thirty, additional changes.

In some embodiments, an anti-CSP antibody of the present invention comprises a V_{L} region having at least 70% identity, at least 75% identity, at least 80% identity, or at least 85% identity, at least 90% identity, or at least 95% identity to SEQ ID NO:2; and having at least one of the following: T at position 20; D or N at position 28; D at position 30; S at position 31; K at position 39; I at position 48; A, T, or Y at position 49; K, Q, S, L, V, or A at position 50; S or N at position 53; D at position 56; Q at position 90, Y at position 92; H at position 94, S or Y at position 95; and W at position 96. In some embodiments, an anti-CSP antibody comprises a V_{L} region that comprises the residue, or the combination of residues, as designated in column 3 of Table 10 in the EXAMPLES section.

### Illustrative Antibodies

In some embodiments, an anti-CSP antibody of the present invention comprises a V_{H} region and a V_{L} region as described in the preceding paragraphs in this section. In some embodiments, the anti-CSP antibody comprises a V_{H} and/or V_{L} region that comprises the residue, or the combination of residues, as designated in column 3 of Table 10 in the EXAMPLES section.

In some embodiments, provided herein is an anti-CSP antibody comprising the CDR1, CDR2, and CDR3 of a V_{H} region of any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95; and/or an anti-CSP antibody comprising the CDR1, CDR2, and CDR3 of a V_{L} region of any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96.

In some embodiments, provided herein are anti-CSP antibodies comprising a V_{H} having at least 90% identity, or at least 95% identity, to an amino acid sequence of any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95. In some embodiments, provided herein are anti-CSP antibodies comprising a V_{L} having at least 90% identity, or at least 95% identity, to an amino acid sequence of any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96.

In some embodiments, an anti-CSP antibody of the present invention comprises a V_{H}comprising an amino acid sequence of any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95; or a V_{L} comprising an amino acid sequence of any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96. In some embodiments, an anti-CSP antibody of the present invention comprises a V_{H} comprising an amino acid sequence of any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95; and a V_{L} comprising an amino acid sequence of any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96.

In some embodiments, an anti-CSP antibody of the present invention comprises a V_{H} having at least 85% identity, or at least 90% identity; or at least 95% identity; and a V_{L} having at least 85% identity, or at least 90% identity; or at least 95% identity to the V_{H} and V_{L} of an antibody as set forth in Table 3. In some embodiments, such an antibody has no more than ten mutations, or no more than nine mutations, no more than eight mutations, or no more than seven mutations in total in the heavy and light chain CDR sequences compared to the CDR sequence of ant antibody as designated in Table 3. In some embodiments, the antibody has six, five, four, three, two or one mutation in total in the heavy and light chain CDR sequences compared to the CDR sequences of an antibody as designated in Table 3. In some embodiments, all of the mutations are substitutions relative to the corresponding sequence shown in Table 3.

TABLE 3
AB-000317 designed variants

| Antibody | | | |
|---|---|---|---|
| Name | Antibody ID | VH amino acid sequence | VL amino acid sequence |
| GermLow | AB-007028 | QMQLVESGGGVVQPGRSL | DIQMTQSPSTLSASVG |
| | | RLSCAASGFTFSTYAMHWV | DRVTITCRASQSISRWL |
| | | RQAPGQGLQWVAVISYHST | AWYQQQPGKAPKLLM |
| | | NKYYEDSVRGRFTISRDNS | SGASVLESGVPSRFSGS |
| | | KNTLYLQMNSLRAEDTAV | GSGTEFTLTISSLQPDD |
| | | YYCARDGYSSSFFDFWGQG | FATYYCQHYNSYFVTF |
| | | TLVTVSS (SEQ ID NO: 3) | GQGTKVEIK (SEQ ID |
| | | | NO: 4) |
| GermMed1 | AB-007029 | QVQLVESGGGVVQPGRSLR | DIQMTQSPSTLSASVG |
| | | LSCTASGFTFSTYAMHWVR | DRVAITCRASQSISRW |
| | | QSPGQGLQWVAVISYHSTN | LAWYQQQPGKAPKLL |
| | | KYYEDSVRGRFTISRDNSK | MSGASVLESGVPSRFS |
| | | NTLYLQMNSLRAEDTAVY | GSGSGTEFTLTISSLQP |
| | | YCARDGYSSSFFDFWGQGT | DDFATYYCQHYNSYF |
| | | LVTVSS (SEQ ID NO: 5) | VTFGQGTKVEIK (SEQ |
| | | | ID NO: 6) |
| GermMed23 | AB-007030 | QMQLVESGGGVVQPGRSL | DIQMTQSPSTLSASVG |
| | | RLSCTASGFTFSTYAMHWV | DRVAITCRASQSISRW |
| | | RQSPGQGLEWVAVISYHST | LAWYQQQPGKAPKLL |
| | | NKYYADSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 7) | VTFGQGTKVEIK (SEQ |
| | | | ID NO: 8) |
| GermMed4 | AB-007031 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLLI |
| | | NKYYEDSVRGRFTISRDNS | SGASVLESGVPSRFSGS |
| | | KNTLYLQMNSLRAEDTAV | GSGTEFTLTISSLQPDD |
| | | YYCARDGYSSSFFDFWGQG | FATYYCQHYNSYFVTF |
| | | TLVTVSS (SEQ ID NO: 9) | GQGTKVEIK (SEQ ID |
| | | | NO: 10) |
| GermLow- | AB-007032 | QVQLVESGGGVVQPGRSLR | DIQMTQSPSTLSASVG |
| Med14 | | LSCAASGFTFSTYAMHWVR | DRVTITCRASQSISRWL |
| | | QAPGQGLQWVAVISYHSTN | AWYQQQPGKAPKLLIS |
| | | KYYEDSVRGRFTISRDNSK | GASVLESGVPSRFSGS |
| | | NTLYLQMNSLRAEDTAVY | GSGTEFTLTISSLQPDD |
| | | YCARDGYSSSFFDFWGQGT | FATYYCQHYNSYFVTF |
| | | LVTVSS (SEQ ID NO: 11) | GQGTKVEIK (SEQ ID |
| | | | NO: 12) |
| GermLow- | AB-007033 | QMQLVESGGGVVQPGRSL | DIQMTQSPSTLSASVG |
| Med23 | | RLSCAASGFTFSTYAMHWV | DRVTITCRASQSISRWL |
| | | RQAPGQGLEWVAVISYHST | AWYQQQPGKAPKLLM |
| | | NKYYADSVRGRFTISRDNS | SGASVLESGVPSRFSGS |
| | | KNTLYLQMNSLRAEDTAV | GSGTEFTLTISSLQPDD |
| | | YYCARDGYSSSFFDFWGQG | FATYYCQHYNSYFVTF |
| | | TLVTVSS (SEQ ID NO: 13) | GQGTKVEIK (SEQ ID |
| | | | NO: 14) |
| GermLow- | AB-007034 | QVQLVESGGGVVQPGRSLR | DIQMTQSPSTLSASVG |
| Med1234 | | LSCAASGFTFSTYAMHWVR | DRVTITCRASQSISRWL |
| | | QAPGQGLEWVAVISYHSTN | AWYQQQPGKAPKLLIS |
| | | KYYADSVRGRFTISRDNSK | GASVLESGVPSRFSGS |
| | | NTLYLQMNSLRAEDTAVY | GSGTEFTLTISSLQPDD |
| | | YCARDGYSSSFFDFWGQGT | FATYYCQHYNSYFVTF |
| | | LVTVSS (SEQ ID NO: 15) | GQGTKVEIK (SEQ ID |
| | | | NO: 16) |
| GermCDR_ | AB-007035 | QMQLVESGGGVVQPGRSL | DIQMTQSPSTLSASVG |
| H31TS | | RLSCTASGFTFSSYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 17) | VTFGQGTKVEIK (SEQ |
| | | | ID NO: 18) |
| GermCDR_ | AB-007036 | QMQLVESGGGVVQPGRSL | DIQMTQSPSTLSASVG |
| H54HD | | RLSCTASGFTFSTYAMHWV | DRVAITCRASQSISRW |
| | | RQSPGQGLQWVAVISYDST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 19) | VTFGQGTKVEIK (SEQ |
| | | | ID NO: 20) |
| GermCDR_ | AB-007037 | QMQLVESGGGVVQPGRSL | DIQMTQSPSTLSASVG |
| H65RK | | RLSCTASGFTFSTYAMHWV | DRVAITCRASQSISRW |
| | | RQSPGQGLQWVAVISYHST | LAWYQQQPGKAPKLL |
| | | NKYYEDSVKGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 21) | VTFGQGTKVEIK (SEQ |
| | | | ID NO: 22) |
| GermCDR_ | AB-007038 | QMQLVESGGGVVQPGRSL | DIQMTQSPSTLSASVG |
| H108FY | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDYWGQ | DDFATYYCQHYNSYF |
| | | GTLVTVSS (SEQ ID NO: 23) | VTFGQGTKVEIK (SEQ |
| | | | ID NO: 24) |
| LiabHydro_ | AB-007039 | QMQLVESGGGVVQPGRSL | DIQMTQSPSTLSASVG |
| L95FS | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYS |
| | | TLVTVSS (SEQ ID NO: 25) | VTFGQGTKVEIK (SEQ |
| | | | ID NO: 26) |
| LiabHydro_ | AB-007040 | QMQLVESGGGVVQPGRSL | DIQMTQSPSTLSASVG |
| L95FY | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYY |
| | | TLVTVSS (SEQ ID NO: 95) | VTFGQGTKVEIK (SEQ |
| | | | ID NO: 96) |
| LiabCharge_ | AB-007041 | QMQLVESGGGVVQPGRSL | DIQMTQSPSTLSASVG |
| H31TD | | RLSCTASGFTFSDYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 27) | VTFGQGTKVEIK (SEQ |
| | | | ID NO: 28) |
| LiabCharge_ | AB-007042 | QMQLVESGGGVVQPGRSL | DIQMTQSPSTLSASVG |
| L28SD | | RLSCTASGFTFSTYAMHWV | DRVAITCRASQDISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 29) | VTFGQGTKVEIK (SEQ |
| | | | ID NO: 30) |
| LiabCharge_ | AB-007043 | QMQLVESGGGVVQPGRSL | DIQMTQSPSTLSASVG |
| L565D | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLEDGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 31) | VTFGQGTKVEIK (SEQ |
| | | | ID NO: 32) |
| LiabDS_ | AB-007044 | QMQLVESGGGVVQPGRSL | DIQMTQSPSTLSASVG |
| H62DE | | RLSCTASGFTFSTYAMHWV | DRVAITCRASQSISRW |
| | | RQSPGQGLQWVAVISYHST | LAWYQQQPGKAPKLL |
| | | NKYYEESVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 33) | VTFGQGTKVEIK (SEQ |
| | | | ID NO: 34) |
| LiabDG_ | AB-007045 | QMQLVESGGGVVQPGRSL | DIQMTQSPSTLSASVG |
| H100GA | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDAYSSSFFDFWGQG | DDFATYYCQHYNSYF |
| | | TLVTVSS (SEQ ID NO: 35) | VTFGQGTKVEIK (SEQ |
| | | | ID NO: 36) |
| LiabNS_ | AB-007046 | QMQLVESGGGVVQPGRSL | DIQMTQSPSTLSASVG |
| L92NY | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYYSYF |
| | | TLVTVSS (SEQ ID NO: 37) | VTFGQGTKVEIK (SEQ |
| | | | ID NO: 38) |
| K_{D}_H31TN | AB-007047 | QMQLVESGGGVVQPGRSL | DIQMTQSPSTLSASVG |
| | | RLSCTASGFTFSNYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 39) | VTFGQGTKVEIK (SEQ |
| | | | ID NO: 40) |
| K_{D}_H33A5 | AB-007048 | QMQLVESGGGVVQPGRSL | DIQMTQSPSTLSASVG |
| | | RLSCTASGFTFSTYSMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 41) | VTFGQGTKVEIK (SEQ |
| | | | ID NO: 42) |
| K_{D}_ | AB-007049 | QMQLVESGGGVVQPGRSL | DIQMTQSPSTLSASVG |
| H53YN + | | RLSCTASGFTFSTYAMHWV | DRVAITCRASQSISRW |
| H55SE | | RQSPGQGLQWVAVISNHET | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 43) | VTFGQGTKVEIK (SEQ |
| | | | ID NO: 44) |
| K_{D}_H53YQ | AB-007050 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISQHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 45) | VTFGQGTKVEIK (SEQ |
| | | | ID NO: 46) |
| K_{D}_H56TR | AB-007051 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHSR | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 47) | VTFGQGTKVEIK (SEQ |
| | | | ID NO: 48) |
| K_{D}_H56TN | AB-007052 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHSN | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 49) | VTFGQGTKVEIK (SEQ |
| | | | ID NO: 50) |
| K_{D}_H57NQ | AB-007053 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | QKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 51) | VTFGQGTKVEIK (SEQ |
| | | | ID NO: 52) |
| K_{D}_H102SA | AB-007054 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYASSFFDFWGQ | DDFATYYCQHYNSYF |
| | | GTLVTVSS (SEQ ID NO: 53) | VTFGQGTKVEIK (SEQ |
| | | | ID NO: 54) |
| K_{D}_H102ST | AB-007055 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYTSSFFDFWGQ | DDFATYYCQHYNSYF |
| | | GTLVTVSS (SEQ ID NO: 55) | VTFGQGTKVEIK (SEQ |
| | | | ID NO: 56) |
| K_{D}_H102SQ | AB-007056 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYQSSFFDFWGQ | DDFATYYCQHYNSYF |
| | | GTLVTVSS (SEQ ID NO: 57) | VTFGQGTKVEIK (SEQ |
| | | | ID NO: 58) |
| K_{D}_H102SH | AB-007057 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYHSSFFDFWGQ | DDFATYYCQHYNSYF |
| | | GTLVTVSS (SEQ ID NO: 59) | VTFGQGTKVEIK (SEQ |
| | | | ID NO: 60) |
| K_{D}_H103ST | AB-007058 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSTSFFDFWGQ | DDFATYYCQHYNSYF |
| | | GTLVTVSS (SEQ ID NO: 61) | VTFGQGTKVEIK (SEQ |
| | | | ID NO: 62) |
| K_{D}_H104ST | AB-007059 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSTFFDFWGQ | DDFATYYCQHYNSYF |
| | | GTLVTVSS (SEQ ID NO: 63) | VTFGQGTKVEIK (SEQ |
| | | | ID NO: 64) |
| K_{D}_H105FY | AB-007060 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSYFDFWGQ | DDFATYYCQHYNSYF |
| | | GTLVTVSS (SEQ ID NO: 65) | VTFGQGTKVEIK (SEQ |
| | | | ID NO: 66) |
| K_{D}_L30SD | AB-007061 | QMQLVESGGGVVQPGRSL | DIQMTQSPSTLSASVG |
| | | RLSCTASGFTFSTYAMHWV | DRVAITCRASQSIDRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 67) | VTFGQGTKVEIK (SEQ |
| | | | ID NO: 68_ |
| K_{D}_L31RS | AB-007062 | QMQLVESGGGVVQPGRSL | DIQMTQSPSTLSASVG |
| | | RLSCTASGFTFSTYAMHWV | DRVAITCRASQSISSWL |
| | | RQSPGQGLQWVAVISYHST | AWYQQQPGKAPKLLM |
| | | NKYYEDSVRGRFTISRDNS | SGASVLESGVPSRFSGS |
| | | KNTLYLQMNSLRAEDTAV | GSGTEFTLTISSLQPDD |
| | | YYCARDGYSSSFFDFWGQG | FATYYCQHYNSYFVTF |
| | | TLVTVSS (SEQ ID NO: 69) | GQGTKVEIK (SEQ ID |
| | | | NO: 70) |
| K_{D}_L495A | AB-007063 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MAGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 71) | VTFGQGTKVEIK (SEQ |
| | | | ID NO: 72) |
| K_{D}_L495T | AB-007064 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MTGASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 73) | VTFGQGTKVEIK (SEQ |
| | | | ID NO: 74) |
| K_{D}_L50GK | AB-007065 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSKASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 75) | VTFGQGTKVEIK (SEQ |
| | | | ID NO: 76) |
| K_{D}_L50GQ | AB-007066 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSQASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 77) | VTFGQGTKVEIK (SEQ |
| | | | ID NO: 78) |
| K_{D}_L50GS | AB-007067 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSSASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 79) | VTFGQGTKVEIK (SEQ |
| | | | ID NO: 80) |
| K_{D}_L50GL | AB-007068 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSLASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 81) | VTFGQGTKVEIK (SEQ |
| | | | ID NO: 82) |
| K_{D}_L50GV | AB-007069 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSVASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 83) | VTFGQGTKVEIK (SEQ |
| | | | ID NO: 84) |
| K_{D}_L50GA | AB-007070 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSAASVLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 85) | VTFGQGTKVEIK (SEQ |
| | | | ID NO: 86) |
| K_{D}_L53V5 | AB-007071 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASSLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 87) | VTFGQGTKVEIK (SEQ |
| | | | ID NO: 88) |
| K_{D}_L53VN | AB-007072 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASNLESGVPSRFS |
| | | KNTLYLQMNSLRAEDTAV | GSGSGTEFTLTISSLQP |
| | | YYCARDGYSSSFFDFWGQG | DDFATYYCQHYNSYF |
| | | TLVTVSS (SEQ ID NO: 89) | VTFGQGTKVEIK (SEQ |
| | | | ID NO: 90) |
| K_{D}_L94YH | AB-007073 | QMQLVESGGGVVQPGRSL | DIQMTQSPSTLSASVG |
| | | RLSCTASGFTFSTYAMHWV | DRVAITCRASQSISRW |
| | | RQSPGQGLQWVAVISYHST | LAWYQQQPGKAPKLL |
| | | NKYYEDSVRGRFTISRDNS | MSGASVLESGVPSRFS |
| | | KNTLYLQMNSLRAEDTAV | GSGSGTEFTLTISSLQP |
| | | YYCARDGYSSSFFDFWGQG | DDFATYYCQHYNSHF |
| | | TLVTVSS (SEQ ID NO: 91) | VTFGQGTKVEIK (SEQ |
| | | | ID NO: 92) |
| GermLow_ | AB-007074 | QMQLVESGGGVVQPGRSL | DIQMTQSPSTLSASVG |
| 5FY | | RLSCAASGFTFSTYAMHWV | DRVTITCRASQSISRWL |
| L9 | | RQAPGQGLQWVAVISYHST | AWYQQQPGKAPKLLM |
| | | NKYYEDSVRGRFTISRDNS | SGASVLESGVPSRFSGS |
| | | KNTLYLQMNSLRAEDTAV | GSGTEFTLTISSLQPDD |
| | | YYCARDGYSSSFFDFWGQG | FATYYCQHYNSYYVT |
| | | TLVTVSS (SEQ ID NO: 93) | FGQGTKVEIK (SEQ ID |
| | | | NO: 94) |

Notes:
the naming of each mutation contains information indicating whether it is located in heavy chain (“H”) or light chain (“L”), the position of the residue, the residues before and after the mutation at the position. For example, “H31TS” represents that Threonine (T) at position 31 of the heavy chain has been replaced with Serine (S).
“GermLow” and “GermMed” refer to variants in which at least one residue in the FWs has been mutated to the corresponding residue in the germline sequence
“GermCDR” refers to variants in which at least one residue in the CDRs has been mutated to the corresponding residue in the germline sequence
“LiabCharge” refers to variants in which at least one residue has been mutated to remove development liabilities associated with charge.
“LiabHydro” refers to variants in which at least one residue has been mutated to remove development liabilities associated with hydrophobicity.

In a further aspect of the invention, an anti-CSP antibody according to any of the above embodiments is a monoclonal antibody, including a chimeric, antibody. In one embodiment, an anti-CSP antibody is an antibody fragment, e.g., a Fv, Fab, Fab′, scFv, diabody, or F(ab′)₂ fragment. In another embodiment, the antibody is a substantially full length antibody, e.g., an IgG antibody or other antibody class or isotype as defined herein. For a review of certain antibody fragments, see Hudson et al. Nat. Med. 9: 129-134 (2003). Antibody fragments can be made by various techniques, including but not limited to proteolytic digestion of an intact antibody as well as production by recombinant host cells (e.g. E. coli or phage), as described herein.

In some embodiments an anti-CSP antibody in accordance with the present disclosure is in a monovalent format. In some embodiments, the anti-CSP antibody is in a fragment format, e.g., a Fv, Fab, Fab′, scFv, diabody, or F(ab′)₂ fragment.

In some embodiments, an anti-CSP antibody of the present invention is employed in a bispecific or multi-specific format. For example, in some embodiments, the antibody may be incorporated into a bispecific or multi-specific antibody that comprises a further binding domain that binds to the same or a different antigen.

In some embodiments, an antibody of the present disclosure comprises an Fc region that has effector function, e.g., exhibits antibody-dependent cellular cytotoxicity ADCC. In some embodiments, the Fc region may be an Fc region engineered to alter one or more functional properties of the antibody, such as serum half-life, complement fixation, Fc receptor binding, and/or ADCC. Furthermore, an antibody of the disclosure may be chemically modified (e.g., one or more chemical moieties can be attached to the antibody) or be modified to alter its glycosylation, again to alter one or more functional properties of the antibody. Additional modifications may also be introduced. For example, the antibody can be linked to one of a variety of polymers, for example, polyethylene glycol.

### Activity

The activity of an anti-CSP antibody variant as described herein can be assessed for binding to CSP, either binding to a series of linear peptides with varying lengths representing the immunodominant regions of the CSP protein or to the entire CSP protein; and/or the ability to protect against challenge with Plasmodium that comprises P. falciuparum CSP, e.g., in in vivo animal models of malaria. In some embodiments, effector function, e.g., ADCC, is also evaluated.

In some embodiments, binding activity of a variant anti-CSP antibody as described herein to P. falciparum CSP protein can be assessed. Binding can be determined using any assay that measures binding to CSP, e.g., surface plasmon resonance (SPR) analysis using a biosensor system or bio-layer interferometry (BLI). Systems suitable for use in SPR are well known and commercially available, for example, LSA™ (Carterra, Dublin, Calif.), Biacore™ (General Electric, Boston, Mass.), and OpenSPR (Nicoya, East Kitchener, ON, Canada). Systems suitable for use in BLI include, but are not limited to, Octet™ (ForteBio, Fremont, Calif.) and Gator™ (Probelife, Palo Alto, Calif.). In an exemplary SPR assay, each antibody can be either directly immobilized to a Carterra CMD200M Chip or captured to the CMD200M Carterra Chip with a goat anti-human IgG Fc antibody. The uncoupled antibodies can be washed off and various concentration gradients of the targets can be flowed over the antibodies. In some cases the highest concentration of each target can be in the range 0.5-8 μg/mL. For better accuracy, each antibody can be immobilized in different locations (e.g., at least 2) on the chip and the affinity for each antibody-target combination can be determined using multiple (e.g., 4-5) target concentrations according to standard methods. In some cases, if variation between the two duplicates is >3-fold, the antibody-target measurement is repeated. For BLI, each of the antigens (e.g., those disclosed in Table 1) can be immobilized on sensors according to manufacture's instructions. In one illustrative example, the antigen can be biotinylated and immobilized to streptavidin sensors. For better accuracy, each antibody can be evaluated in replicates at a suitable concentration (e.g., 5 μg/mL). In some cases, if variation between the two duplicates is >3-fold, the antibody-target measurement is repeated. The assays are typically performed under conditions according to manufacture's instructions. In some cases, the assays are performed under a temperature in the range of 20° C. to 37° C., for example 20° C.-25° C. In one embodiment, the assay is performed at 25° C. In one embodiment, the assay is performed at 37° C.

In some embodiments, binding to CSP protein is assessed in a competitive assay format with a reference antibody AB-000317 or a reference antibody having the variable regions of AB-000317. In some embodiments, a variant anti-CSP antibody in accordance with the present disclosure may block binding of the reference antibody in a competition assay by about 50% or more.

Anti-CSP antibodies of the present disclosure may also be evaluated in various assays for their ability to mediate FcR-dependent activity. In some embodiments, an antibody of the present disclosure has enhanced ADCC and/or serum stability compared to antibody AB-000317 when the antibodies are assayed in a human IgG1 isotype format.

In some embodiments, activity of an anti-CSP antibody variant is evaluated in vivo in an animal model, e.g., as described in the Examples section. Various assays for measuring activity of anti-CSP antibodies in vivo are known. One exemplary assay is the mouse malaria liver burden assay, as disclosed in Flores-Garcia Y, et al. Malar J. 2019; 18(1):426, doi:10.1186/s12936-019-3055-9, the relevant portion is herein incorporated by reference. In one illustrative example, mice are administered antibody and infected with transgenic P. berghei expressing GFP-luciferase and P. falciparum CSP protein. Parasite liver load can be evaluated, e.g., by RT-qPCR or by measuring bioluminescence with an IVIS Spectrum imager. A reduction in parasite liver load reflects prophylactic activity of an antibody.

A variant as described herein has at least 50%, or at least 60%, or 70%, or greater, of the activity of AB-000317 when evaluated under the same assay conditions. In some embodiments, an anti-CSP antibody exhibits improved activity, i.e., greater than 100%, activity compared to AB-000317. In some embodiments, the anti-CSP antibody variants disclosed herein have similar activity against malaria infection as compared to AB-000317. The term “similar activity,” when used to compare in vivo activity of antibodies, refers to that two measurements of the activity is no more than 30%, no more than 25%, no more than 20%, no more than 15% different, no more than 10%, no more than 8%, or no more than 5% different from each other.

In some embodiments, the native anti-CSP antibody, AB-000317, is modified to have improved developability (i.e., reduced development liabilities), including but not limited to, decreased heterogeneity, increased yield, increased stability, improved net charges to improve pharmacokinetics, and or/reduced immunogenicity. In some embodiments, antibodies having improved developability can be obtained by introducing mutations to reduce or eliminate potential development liabilities, as described in Table 4. In some embodiments, antibodies having improved developability possess modifications as compared to AB-000317 in their amino acid sequence, as disclosed in Table 5.

In some embodiments, the anti-CSP antibody variants disclosed herein have improved developability while maintaining comparable or improved binding affinity to the target as compared to AB-000317. Non-limiting examples of such anti-CSP antibody variants are disclosed in Table 12A and 12B. In some embodiments, the anti-CSP antibody variants disclosed herein have improved developability while maintaining activities that are similar to AB-000317. Non-limiting examples of such anti-CSP antibody variants are described in Table 13.

### Generation of Antibodies

CSP antibodies as disclosed herein are commonly produced using vectors and recombinant methodology well known in the art (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors. Accordingly, in a further aspect of the invention, provided herein are isolated nucleic acids encoding a V_{H} and/or V_{L} region, or fragment thereof, of any of the anti-CSP antibodies as described herein; vectors comprising such nucleic acids and host cells into which the nucleic acids are introduced that are used to replicate the antibody-encoding nucleic acids and/or to express the antibodies. Such nucleic acids may encode an amino acid sequence containing the V_{L} and/or an amino acid sequence containing the V_{H} of the anti-CSP antibody (e.g., the light and/or heavy chains of the antibody). In some embodiments, the host cell contains (1) a vector containing a polynucleotide that encodes the V_{L} amino acid sequence and a polynucleotide that encodes the V_{H} amino acid sequence, or (2) a first vector containing a polynucleotide that encodes the V_{L} amino acid sequence and a second vector containing a polynucleotide that encodes the V_{H} amino acid sequence.

In a further aspect, the invention provides a method of making an anti-CSP antibody as described herein. In some embodiments, the method includes culturing a host cell as described in the preceding paragraph under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1 plasmids, pCR1, RP4, phage DNAs, and shuttle vectors. These and many other cloning vectors are available from commercial vendors.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids and viral vectors, including adenoviruses, adeno-associated viruses, retroviruses, and any other vector.

Suitable host cells for expressing an anti-CSP antibody as described herein include both prokaryotic or eukaryotic cells. For example, anti-CSP antibodies may be produced in bacteria, in particular when glycosylation and Fc effector function are not needed. After expression, the antibody may be isolated from the bacterial cell paste in a soluble fraction and can be further purified. Alternatively, the host cell may be a eukaryotic host cell, including eukaryotic microorganisms, such as filamentous fungi or yeast, including fungi and yeast strains whose glycosylation pathways have been “humanized,” resulting in the production of an antibody with a partially or fully human glycosylation pattern, vertebrate, invertebrate, and plant cells. Examples of invertebrate cells include insect cells. Numerous baculoviral strains have been identified which may be used in conjunction with insect cells. Plant cell cultures can also be utilized as host cells.

In some embodiments, vertebrate host cells are used for producing anti-CSP antibodies of the present disclosure. For example, mammalian cell lines such as a monkey kidney CV1 line transformed by SV40 (COS-7); human embryonic kidney line (293 or 293 cells as described, e.g., in Graham et al., J. Gen Virol. 36:59, 1977; baby hamster kidney cells (BHK); mouse sertoli cells (TM4 cells as described, e.g., in Mather, Biol. Reprod. 23:243-251, 1980 monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68, 1982; MRC 5 cells; and FS4 cells may be used to express anti-CSP antibodies. Other useful mammalian host cell lines include Chinese hamster ovary (CHO) cells, including DHFR-CHO cells (Urlaub et al., Proc. Natl. Acad. Sci. USA 77:4216, 1980); and myeloma cell lines such as Y0, NS0 and Sp2/0. Host cells of the present disclosure also include, without limitation, isolated cells, in vitro cultured cells, and ex vivo cultured cells. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268, 2003.

A host cell transfected with an expression vector encoding an anti-CSP antibody of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

In some embodiments, provided herein is a method of generating variants of an anti-CSP antibody as disclosed herein. Thus, for example, a construct encoding a variant V_{H} CDR3 as described in the “anti-CSP Antibody Variant” section can be additionally modified and the V_{H} region encoded by the additionally modified construct can be tested for binding activity to CSP and/or in vivo protective efficacy in the context of a V_{H} region comprising the native AB-000317 CDR1 and CDR2, or a variant CDR1 or CDR2 as described herein, that is paired with a native AB-000317 V_{L} region or variant region as described herein. Similarly, a construct encoding a variant V_{L} CDR3 as described in the “anti-CSP Antibody Variant” section can be additionally modified and the V_{L} region encoded by the additionally modified construct can be tested for binding activity to CSP and/or protective efficacy. Such an analysis can also be performed with other CDRs or framework regions and an antibody having the desired activity can then be selected.

### Anti-CSP Antibody Conjugates

In a further aspect, an anti-CSP antibody of the present invention may be conjugated or linked to therapeutic and/or imaging/detectable moieties. For example, the anti-CSP antibody may be conjugated to a detectable marker, a toxin, or a therapeutic agent. Methods for conjugating or linking antibodies are well known in the art. The moiety may be linked to the antibody covalently or by non-covalent linkages.

In some embodiments, the antibody is conjugated to cytotoxic moiety or other moiety that inhibits cell proliferation. In some embodiments, the antibody is conjugated to a cytotoxic agent including, but not limited to, a ricin A chain, doxorubicin, daunorubicin, a maytansinoid, taxol, ethidium bromide, mitomycin, etoposide, tenoposide, vincristine, vinblastine, colchicine, dihydroxy anthracin dione, actinomycin, a diphtheria toxin, extotoxin A from Pseudomonas, Pseudomonas exotoxin (PE) A, PE40, abrin, abrin A chain, modeccin A chain, alpha sarcin, gelonin, mitogellin, restrictocin, cobran venom factor, a ribonuclease, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In some embodiments, the antibody may be linked to an agent such as an enzyme inhibitor, a roliferation inhibitor, a lytic agent, a DNA or RNA synthesis inhibitors, a membrane permeability modifier, a DNA metabolites, a dichloroethylsulfide derivative, a protein production inhibitor, a ribosome inhibitor, or an inducer of apoptosis.

In some embodiments, the antibody may be linked to radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

In some embodiments, the present invention features bispecific molecules comprising an anti-CSP antibody, or a fragment thereof, of the invention. The anti-CSP antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The anti-CSP antibody of the invention may, in fact, be derivatized or linked to more than one other functional molecule to generate multispecific molecules that bind to more than two different binding sites (e.g., two different epitopes on the CSP protein) and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results. In one illustrative embodiment, the bispecific antibody can be created using the knobs-into-holes strategy. The strategy typically involves first creating a first half of the antibody that recognizes a first antigen, e.g., CSP, and a second half of the antibody that recognizes a second antigen or binding site, and then joining the two halves to create the bispecific antibody. In some embodiments, the first antigen and the second antigen are different epitopes of the CSP protein.

### Pharmaceutical Compositions

In a further aspect, provided herein are pharmaceutical compositions for administration of an anti-CSP antibody of the present invention to a mammalian subject, preferably a human, who has malaria or is at risk for malaria, in an amount and according to a schedule sufficient to prevent Plasmodium infection, e.g., infection with Plasmodium falciparum or a Plasmodium sp. having a cross-reactive CSP protein, or to reduce a symptom of malaria in the subject. Such compositions may comprise an anti-CSP antibody as described herein, or a polynucleotide encoding the antibody, and a pharmaceutically acceptable diluent or carrier. In some embodiments, a polynucleotide encoding the antibody may be contained in a plasmid vector for delivery, or a viral vector. In some embodiments, the pharmaceutical composition comprises a therapeutically effective amount of the antibody. As used herein, a “therapeutically effective dose” or a “therapeutically effective amount” refers to an amount sufficient to prevent, cure, or at least partially arrest malaria or symptoms of malaria. A therapeutically effective dose can be determined by monitoring a patient's response to therapy. Typical benchmarks indicative of a therapeutically effective dose include amelioration or prevention of symptoms of malaria in the patient, including, for example, reduction in the number of parasites. Amounts effective for this use will depend upon the severity of the disease and the general state of the patient's health, including other factors such as age, weight, gender, administration route, etc. Single or multiple administrations of the antibody will be dependent on the dosage and frequency as required and tolerated by the patient.

In some embodiments, antibody is administered at a pre-erythrocyte stage of infection, i.e., antibody is administered in a time frame to prevent or reduce hepatocyte infection.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in Remington: The Science and Practice of Pharmacy 20th Ed. (Lippincott, Williams & Wilkins 2012). In particular embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., phosphate-buffered saline; sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The pharmaceutical composition can be formulated for any suitable route of administration, including for example, parenteral, intrapulmonary, intranasal, or local administration. Parenteral administration can include intramuscular, intravenous, intraarterial, intraperitoneal, oral or subcutaneous administration. In certain embodiments, the pharmaceutical composition is formulated for intravenous administration and has a concentration of antibody of 10-100 mg/ml, 10-50 mg/ml, 20 to 40 mg/ml, or about 30 mg/ml. In certain embodiments, the pharmaceutical composition is formulated for subcutaneous injection and has a concentration of antibody of 50-500 mg/ml, 50-250 mg/ml, or 100 to 150 mg/ml, and a viscosity less than 50 cP, less than 30 cP, less than 20 cP, or about 10 cP. In some embodiments, the pharmaceutical compositions are liquids or solids. In particular embodiments, the pharmaceutical compositions are formulated for parenteral, e.g., intravenous, subcutaneous, intraperiotoneal, or intramuscular administration.

The formulation of and delivery methods of pharmaceutical compositions will generally be adapted according to the site and the disease to be treated. Formulations include those in which the antibody is encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays.

In some embodiments, e.g., for parenteral administration, the antibodies or antigen-binding fragments thereof are formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate may also be used.

The dose and dosage regimen depends upon a variety of factors readily determined by a physician, such as the nature of the infection, the characteristics of the subject, and the subject's history. In particular embodiments, the amount of antibody or antigen-binding fragment thereof administered or provided to the subject is in the range of about 0.1 mg/kg to about 50 mg/kg of the subject's body weight. Depending on the type and severity of the infection, in certain embodiments, about 0.1 mg/kg to about 50 mg/kg body weight (e.g., about 0.1-15 mg/kg/dose) of antibody or antigen-binding fragment thereof may be provided as an initial candidate dosage to the patient, whether, for example, by one or more separate administrations, or by continuous infusion. The progress of the therapy is readily monitored by conventional methods and assays and based on criteria known to the physician or other persons of skill in the art.

An antibody of the present disclosure may be administered to a subject using any route of administration, e.g., systemic, parenterally, locally, in accordance with known methods. Such routes include, but are not limited to, intravenous administration, e.g., as a bolus or by continuous infusion over a period of time, by intramuscular, intraperitoneal, intracerobrospinal, subcutaneous, intraarticular, intrasynovial, intrathecal, oral, topical, or inhalation routes. A subject may be administered an antibody of the present invention one or more times; and may be administered before, after, or concurrently with another therapeutic agent as further described below.

In certain embodiments, the antibody is provided to the subject in combination with one or more additional therapeutic agents used to treat or prevent malaria or a related disease or disorder. In certain embodiments, a method for treating or preventing malaria is provided, comprising administering to the human a therapeutically effective amount of an antibody as disclosed herein, or a pharmaceutically acceptable salt thereof, in combination with a therapeutically effective amount of one or more additional therapeutic agents. In one embodiment, a method for treating malaria in a human having or at risk of having the infection is provided, comprising administering to the human a therapeutically effective amount of an antibody as disclosed herein, or a pharmaceutically acceptable salt thereof, in combination with a therapeutically effective amount of one or more additional therapeutic agents.

In certain embodiments, when an antibody of the present disclosure as described herein is combined with one or more additional therapeutic agents as described above, the components of the composition are administered as a simultaneous or sequential regimen. When administered sequentially, the combination may be administered in two or more administrations.

In some embodiments, an antibody as disclosed herein is combined with one or more additional therapeutic agents in a unitary dosage form for simultaneous administration to a patient.

A “patient” refers to any subject receiving the antibody regardless of whether they have malaria. In some embodiments, a “patient” is a non-human subject, e.g., an animal that is used as a model of evaluating the effects of antibody administration.

“Co-administration” of an antibody as disclosed herein with one or more additional therapeutic agents generally refers to simultaneous or sequential administration of an antibody or fragment thereof disclosed herein and one or more additional therapeutic agents, such that therapeutically effective amounts of the antibody or fragment thereof disclosed herein and one or more additional therapeutic agents are both present in the body of the patient.

Co-administration includes administration of unit dosages of the antibody disclosed herein before or after administration of unit dosages of one or more additional therapeutic agents, for example, administration of the antibody within seconds, minutes, or hours of the administration of one or more additional therapeutic agents. For example, in some embodiments, a unit dose of an antibody disclosed herein is administered first, followed within seconds or minutes by administration of a unit dose of one or more additional therapeutic agents.

Alternatively, in other embodiments, a unit dose of one or more additional therapeutic agents is administered first, followed by administration of a unit dose of an antibody within seconds or minutes. In some embodiments, a unit dose of an antibody disclosed herein is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of the antibody.

The combined administration may be co-administration, using separate pharmaceutical compositions or a single pharmaceutical composition, or consecutive administration in either order, wherein there is optionally a time period while both (or all) therapeutic agents simultaneously exert their biological activities. Such combined therapy may result in a synergistic therapeutic effect. In certain embodiments, it is desirable to combine administration of an antibody of the invention with another antibody directed against another Plasmodium falciparum antigen, or against a different CSP target epitope.

As described herein, the antibody may also be administered by gene therapy via a nucleic acid comprising one or more polynucleotides encoding the antibody. In certain embodiments, the polynucleotide encodes an scFv. In particular embodiments, the polynucleotide comprises DNA, cDNA or RNA. In certain embodiments, the polynucleotide is present in a vector, e.g., a viral vector.

The following examples are offered for illustrative purposes, and are not intended to limit the invention. Those of skill in the art will readily recognize a variety of non-critical parameters that can be changed or modified to yield essentially the same results.

### EXAMPLES

### Example 1. Generation of Anti-CSP Antibody Variants

Antibody AB-000317 was obtained from a donor enrolled in a Phase 2a study evaluating efficacy of the RTS,S vaccine in preventing malaria infection. The RTS,S vaccine is a pseudo-viral particle vaccine that combines the hepatitis B surface antigen and the central repeat and C-terminal regions of the CSP protein. All study participants were vaccinated with one of two vaccine schedules (standard full-dose: 0, 1, 2 M or fractional-third dose: 0, 1, 7 M), or placebo and subsequently challenged with a controlled human malaria parasite infection. The donor from whom AB-000317 was identified was protected following challenge. Heavy and light chain AB-000317 sequences were expressed as a human IgG1 monoclonal antibody. Compared to other antibodies obtained from the same or different donors, AB-000317 demonstrated strong binding and affinity to CSP protein in vitro, no binding to Hepatitis B protein, and exceptional functional activity when tested in vivo. The sequence pair encoding for AB-000317 was selected as the most frequently observed plasmablast (n=28) from one of the most dominant lineages (59 siblings in total), where a lineage is defined as 2 or more sequence pairs predicted to have originated from the same VH and VL germline genes and have H-CDR3 sequences and L-CDR3 sequences of identical length. A “lineage” represents a set of antibodies that are derived from a common progenitor B cell and a single recombination event.

AB-000317 demonstrated binding to CSP when evaluated at 2.5 μg/mL and 0.15 μg/mL. Additional mapping studies refined the AB-0317 epitope to the CSP (NPNA)₃ region and determined a K_{D} of 0.078 μM±16 when evaluated for affinity (Oyen et al., Proc. Natl. Acad Sci. USA 114:E10438-E10445, 2017; Epub Nov. 14, 2017). AB-000317 was also evaluated in vivo using a murine malaria infection model. In this model mice were administered 300 μg mAb and intravenously (iv) challenged 5-10 min later with P. berghei engineered to express full-length P. falciparum CSP. Parasitic liver load was evaluated 40 hours post-infection by quantitative PCR (qPCR). Mice administered 300 μg of AB-000317 demonstrated a 99.7% reduction in parasite liver load as compared to non-treated, infected control mice (Oyen et al., supra). AB-000317 has also been evaluated in complex with (NPNA)₃ at 2.4 Å resolution resulting in a refined structural model of the complexed antibody-antigen (Oyen et al., supra).

This example describes the design of improved variants of AB-000317. In one aspect, the variants generated have improved developability, e.g., as identified through various in vitro assays, such as aggregation assessment by HPLC or UPLC, hydrophobic interaction chromatography (HIC), polyspecificity assays (e.g., baculovirus particle binding), self-interaction nanoparticle spectroscopy (SINS), or mass spec analysis after incubation in an accelerated degradation condition such as high temperature, low pH, high pH, or oxidative H₂O₂. Mutations are successful if activity is maintained (or enhanced) while removing or reducing the severity of the liability.

Improved properties of antibody variants generated as described in this example include: (1) fits a standard platform (expression, purification, formulation); (2) high yield; (3) low heterogeneity (glycosylation, chemical modification, etc.); (4) consistent manufacturability (batch-to-batch, and small-to-large scale); (5) high stability (years in liquid formulation), e.g., minimal chemical degradation, fragmentation, and aggregation; and (6) long PK (in vivo half life), e.g., no off-target binding, no impairment of FcRn recycling, and stable. Antibody liabilities are further described in Table 4.

TABLE 4
Description of potential development liabilities

| | | sequence | |
|---|---|---|---|
| | | comprises an | |
| | Yield, heterogeneity, | odd number of | |
| Free cysteine⁶ | stability, activity | cysteines | High |
| N-linked | Yield, heterogeneity, | N(~P)(S, T)¹ | High |
| glycosylation | activity | | |
| Abnormal net | Platform fit, PK | Sharma 2014² | High |
| charge | | | |
| Patches of | Stability, PK | Sharma 2014 | High |
| hydro- | | | |
| phobicity | | | |
| Patches of | Stability, PK | N/A (based on | Medium |
| same charge | | structure) | |
| Proteolysis | Stability, PK | (K, R)(K, R)³ | Medium |
| Proteolysis | Stability, PK | DP | Medium |
| Asparagine | Heterogeneity, stability, | NG; | Medium; |
| deamidation | activity | N(A, N, S, T)⁴ | Low |
| Aspartate | Heterogeneity, stability, | DG; | Medium; |
| isomerization | activity | D(A, D, S, T)⁵ | Low |
| Lysine | Heterogeneity, stability, | K | Low |
| glycation | activity | | |
| Methionine | Heterogeneity, stability, | M | Low |
| oxidation | activity | | |
| Tryptophan | Heterogeneity, stability, | W | Low |
| oxidation | activity | | |

Note:
¹The N-linked glycosylation site is N-X-S/T, where X is any residue other than proline.
²Sharma et al., Proc. Natl. Acad. Sci. USA 111: 18601-18606, 2014
³This motif consists of a K or R, followed by a K or R. Stated differently, the motif can be KK, KR, RK, or RR.
⁴The dipeptide NG poses a medium risk of development liability. The dipeptides NA, NN, NS, and NT pose a low risk of development liability. N may also exhibit low risk of liability for other successor residues, e.g., D, H, or P. Stated differently, dipeptide ND, NH, or NP poses a low risk of development liability.
⁵Similary to the above, the dipeptide DG poses a medium risk of development liability. The dipeptides DA, DD, DS, and DT pose a low risk of development liability. D may also exhibit low risk of development liability for other successor residues, e.g., N, H, or P.
⁶“Free cysteine” refers to a cysteine that does not form a disulfide bond with another cysteine and thus is left “free” as thiols. The presence of free cysteines in the antibody can be a potential development liability. Typically, an odd net number of cysteines in the protein shows a likelihood there is a free cysteine.

Another goal for engineering variants is to reduce the risk of clinical immunogenicity: the generation of anti-drug antibodies against the therapeutic antibody. To reduce risk, the AB-000317 sequences were evaluated to identify residues that can be engineered to increase similarity to the intended population's native immunoglobulin variable region sequences.

The factors that drive clinical immunogenicity can be classified into two groups. First are factors that are intrinsic to the drug, such as: sequence; post-translational modifications; aggregates; degradation products; and contaminants. Second are factors related to how the drug is used, such as: dose level; dose frequency; route of administration; patient immune status; and patient HLA type.

One approach to engineering a variant to be as much like self as possible is to identify a close germline sequence and mutate as many mismatched positions (also known as “germline deviations”) to the germline residue type as possible. This approach applies for germline genes IGHV, IGHJ, IGKV, IGKJ, IGLV, and IGLJ, and accounts for all of the variable heavy (VH) and variable light (VL) regions except for part of H-CDR3. Germline gene IGHD codes for part of the H-CDR3 region but typically exhibits too much variation in how it is recombined with IGHV and IGHJ (e.g., forward or reverse orientation, any of three translation frames, and 5′ and 3′ modifications and non-templated additions) to present a “self” sequence template from a population perspective.

Each germline gene can present as different alleles in the population. The least immunogenic drug candidate, in terms of minimizing the percent of patients with an immunogenic response, would likely be one which matches an allele commonly found in the patient population. Single nucleotide polymorphism (SNP) data from the human genome can be used to approximate the frequency of alleles in the population.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for prediction of HLA binding and in vitro assessment to further identify potential epitopes.

Antibody variants are additionally designed to enhance the efficacy of the antibody. In the present example, design parameters for this aspect focused on CDRs, e.g., CDR3. Positions to be mutated were identified based on structural analysis of antibody-antigen co-crystals (Oyen et al., Proc. Natl. Acad Sci. USA 114:E10438-E10445, 2017; Epub Nov. 14, 2017) and based on sequence information of other antibodies from the same lineage as AB-000317.

### Approaches to Mutation Design

Development liabilities can be removed or reduced by one or more mutations. Mutations are designed to preserve antibody structure and function while removing or reducing development liabilities and to improve function. In one aspect, mutations to chemically similar residues were identified that maintain size, shape, charge, and/or polarity. Illustrative mutations are described in Table 5.

TABLE 5
preferred mutations to remove development liabilities

| | | | → |
|---|---|---|---|
| Free cysteine | Odd # C | High | C→(A, S) |
| N-linked | N(~P)(S, T) | High | N→(Q, D, S, A); |
| glycosylation | | | (S, T)→(A, N) |
| Proteolytic | (K, R)(K, R) | Medium | K, R→(Q, S, A) |
| cleavage | | | |
| Proteolytic | DP | Medium | D→(E, S, A) |
| cleavage | | | |
| Asparagine | NG; | Medium; Low | N→(Q, S, A); G→(A, S) |
| deamidation | N(A, N, S, T)* | | |
| Aspartate | DG; | Medium; Low | D→(E, S, A); G→(A, S) |
| isomerization | D(A, D ,S, T)* | | |
| Lysine | K | Low | K→(R, Q, S, A) |
| glycation | | | |
| Methionine | M | Low | M→(Q, L, S, A) |
| oxidation | | | |
| Tryptophan | W | Low | W→(Y, F) |
| oxidation | | | |

Note:
the last column of Table 5 shows preferred mutations. For example, C→(A, S) refers to that C can be mutated to either A or S in order to remove development liabilities.

### Assessment of AB-000317

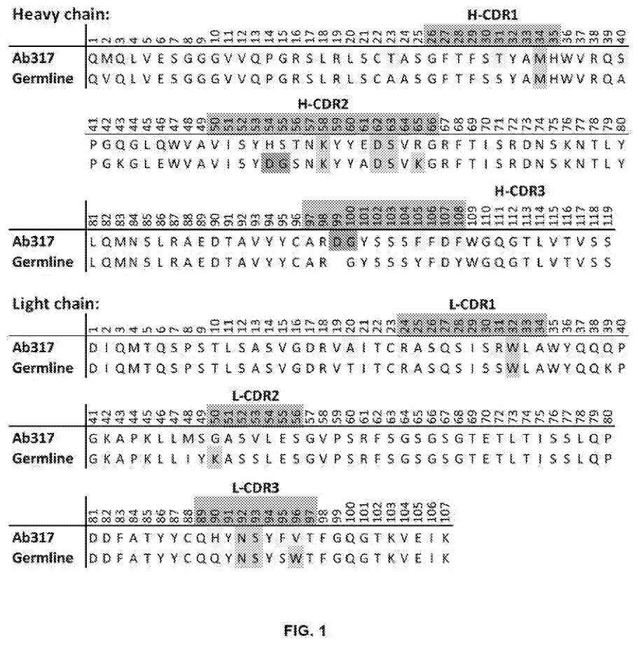

AB-000317 was aligned to the putative V (VH 3-30*01), D (DH 6-13) and J (JH 4) germline genes (FIG. 1). CDRs, germline deviations, and potential liabilities were identified.

Non-canonical cysteines and N-glycosylation sites were identified across the full V_{H} and V_{L}, whereas the other potential liability motifs were identified only within the CDRs.

Potential PK risk was also estimated (Sharma et al., 2014, supra). High hydrophobicity index (HI) was found to correlate with faster clearance, where HI<5 is preferred to reduce risk, and HI<4 is most preferred to reduce risk. However, some antibodies with HI>4, or HI>5, will not exhibit fast clearance. Secondly, too high or too low Fv charge as calculated at pH 5.5 was found to correlate with faster clearance, where charge between (−2, +8) is preferred to reduce risk, and charge between (0, +6.2) is most preferred to reduce risk of fast clearance. Table 6 summarizes the types and number of potential liabilities in AB-000317.

TABLE 6
Summary of potential liabilities in AB-000317

| | AB-000317 |
|---|---|
| Odd # C | 0 |
| N(~P)(S, T) | 0 |
| Fv charge 5.5 | 6.9 |
| HI | 2.9 |
| (K, R)(K, R) | 0 |
| DP | 0 |
| NG | 0 |
| N(A, N, S, T) | 1 |
| DG | 1 |
| D(A, D, S, T) | 1 |
| K | 1 |
| M | 1 |
| W | 1 |
| FW non-germ | 9 |
| VH | 5 |
| VL | 4 |

### Design of Variants to Germline AB-000317

Framework and complementary-determining region (CDR) germline deviations in AB-000317 (as shown in Tables 7 & 8) were analyzed for their potential to be mutated, individually or in combination, to germline sequence, without negatively impacting binding to the (NANP)₃ region of the CSP protein or potency. For each of the candidate mutations from AB-000317 sequence to germline sequence, the risk of making the mutation was assessed based on: (1) the change in charge, if any, since change in charge is intrinsically risky, and a change to more positive charge is particularly risky given the already net positive charge of AB-000317 Fv; (2) conservation of the native AB-000317 residue in the lineage versus the presence of the germline residue or other mutations at that position in the lineage and (3) the structural location of the position with respect to the NANP motif Some mutations were noted to be coupled to at least one other mutation, meaning that the risk prediction is based on making the mutation in conjunction with the other mutation(s). Positions in the V_{H} and V_{L} regions of AB-000317 that can be varied are shown in Tables 7 and 8.

TABLE 7
Assessment of germlining the framework of AB-000317

| | | Germline | | |
|---|---|---|---|---|
| | Change | in a | | |
| Germlining | in | AB-000317 | | |
| mutation | charge | sibling | Location | Description |
| H2MV | 0.0 | Yes | H-FW1; far | borders H98R |
| | | | from NANP | which salt |
| | | | | bridges w/H107D |
| | | | | which could |
| | | | | support H3 |
| | | | | conformation |
| H23TA | 0.0 | Yes | H-FW1; far | Solvent exposed, |
| | | H23E is also | from NANP | away from CDRs |
| | | observed in | | |
| | | sibs | | |
| H40SA | 0.0 | Yes | H-FW2; Base | Base of Fv, |
| | | | of Fv | mostly exposed, |
| | | | | nearby H43QK |
| H43QK | +1.0 | Yes | H-FW2; Base | Base of Fv, |
| | | | of Fv | exposed, nearby |
| | | | | H40SA |
| H46QE | −1.0 | Yes | H-FW2; | Middle of Fv, |
| | | | Middle of Fv | exposed, but |
| | | | | nearby H38R & |
| | | | | H61E. Potential |
| | | | | H61E repulsion, |
| | | | | so can combine |
| | | | | w/H61EA |
| L20AT | 0.0 | Yes | L-FW1; Side | Side of Fv, |
| | | | of Fv | exposed |
| L39QK | +1.0 | Yes | L-FW2; Base | Base of Fv, |
| | | L39H is also | of Fv | exposed, Hbond |
| | | observed in | | w/L81Oxy, near |
| | | sibs. | | L81D, K likely |
| | | | | to interact |
| | | | | w/either |
| L48MI | 0.0 | Yes) | L-FW2; Close | Below L-CDR2, |
| | | | to CDRs | close to CDRs |
| L495Y | 0.0 | No | L-FW2; | Hbond with |
| | | L49A is | Contacting | bound peptide |
| | | observed in | NANP | N3, do not mutate |
| | | 45% of sibs. | | to Y. |

TABLE 8
Assessment of germlining the CDRs of AB-000317

| | | Germline | | |
|---|---|---|---|---|
| | | in a | | |
| Germlining | Change | AB-000317 | | |
| mutation | in charge | sibling | Where | Description |
| H31TS | 0.0 | No | H-CDR1; | Surface, but not |
| | | | Surface | contacting peptide |
| H54HD | −2.0 | No | H-CDR2; | Surface, but not |
| | | | Surface | contacting peptide. |
| | | | | Second shell |
| H555G | 0.0 | No | H-CDR2; | Surface, but not |
| | | | Surface | contacting peptide. |
| | | | | Second shell |
| H56TS | 0.0 | Yes | H-CDR2; | Surface, but not |
| | | | Surface | contacting peptide. |
| | | | | Second shell |
| H61EA | +1.0 | Yes | H-CDR2 | Point down toward |
| | | | | H46Q and H38R. |
| | | | | Combine with |
| | | | | H46QE germlining |
| | | | | mutation |
| H65RK | 0.0 | Yes | H-CDR2; | Surface, but not |
| | | | Surface | contacting peptide. |
| | | | | Salt bridge |
| | | | | to H62D. |
| | | | | Makes low-risk |
| | | | | glycation site |
| H105FY | 0.0 | No | H-CDR3 | |
| H108FY | 0.0 | Yes | H-CDR3 | Seems to fit Y |
| | | | | hydroxyl but also |
| | | | | several nearby |
| | | | | residues of interest |
| L31RS | −1.0 | Yes | L-CDR1 | H bonds to L66oxy |
| | | | | and L52S, nearby |
| | | | | bound peptide and |
| | | | | may lock CDR |
| | | | | conformations |
| L50GK | +1.0 | Yes | L-CDR2 | Near peptide, space |
| | | | | for a side chain. |
| L53V5 | 0.0 | Yes | L-CDR2 | Almost contacts |
| | | | | aliphatic parts of |
| | | | | bound peptide, so |
| | | | | polar S may reduce |
| | | | | binding |
| L90HQ | −1.0 | No | L-CDR3 | Packed under |
| | | | | L-CDR3, which |
| | | | | makes several |
| | | | | contacts to bound |
| | | | | peptide |
| L95FS | 0.0 | No | L-CDR3 | Small hydrophobic |
| | | | | patch from phenyls |
| | | | | of L94Y, L95F, & |
| | | | | H59Y. L94Y & |
| | | | | H59Y contact |
| | | | | peptide; L95F |
| | | | | doesn't. Decreases |
| | | | | HI from 2.94 to 2.36 |
| L96VW | 0.0 | No | L-CDR3 | no space |

Design of Variants to Remove Liabilities from AB-00037

Various sequence-based liabilities in AB-000317 were analyzed for their potential to be mutated to reduce or remove the risk of liability without negatively impacting binding to the (NANP)₃ region of the CSP protein or potency. Residues that contributed to the hydrophobicity index, or to reducing the Fv charge were also assessed. Similar to the germlining design, risk was assessed based on change in charge, shape, polarity, backbone conformation preference, and maintenance or enhancement of side chain interactions (Table 9).

TABLE 9
Design of variants to remove liabilities from AB-000317

| Liability | Liability | | Change in | Mutation | |
|---|---|---|---|---|---|
| position(s) | type | Location | charge | in a sibling | Description |
| L32Wx¹ | Reduce | L-CDR1, | To be | No | Very small |
| | HΦ³ | contact | determined | | hydrophobic patch |
| | | with | | | from the exposed |
| | | peptide | | | side of this W. |
| | | | | | However, lots of |
| | | | | | peptide contact |
| L94YH | Reduce | L-CDR3 | +1.0 | No | H may make new |
| | HΦ, | | | | Hbond to peptide |
| | Improve | | | | backbone Oxy. |
| | K_{D} | | | | Also, decreases |
| | | | | | small patch, HI |
| | | | | | from 2.94 to 2.66 |
| L95F5 | Reduce | L-CDR3 | 0.0 | No | Small hydrophobic |
| | HΦ, | | | | patch from phenyls |
| | Germline | | | | of L94Y, L95F, & |
| | | | | | H59Y. L94Y & |
| | | | | | H59Y contact |
| | | | | | peptide; L95F |
| | | | | | doesn't. Decreases |
| | | | | | HI from 2.94 to |
| | | | | | 2.36 |
| L95FY | Reduce | L-CDR3 | 0.0 | Yes | Decreases HI from |
| | HΦ | | | | 2.94 to 2.53 |
| H23TE | Reduce q | H-FW1 | −1.0 | Yes | Solvent exposed, |
| | | | | | away from CDRs. |
| | | | | | From non-germline |
| | | | | | to other non- |
| | | | | | germline |
| H30SD | Reduce q | H-CDR1, | −1.0 | No | Surface, but not |
| | | Surface | | | contacting peptide. |
| | | exposed | | | N in 1 sibling. |
| | | | | | Would make a DT |
| | | | | | site. Goes away |
| | | | | | from germline |
| H31TD | Reduce q | H-CDR1, | −1.0 | Yes | Surface, but not |
| | | Surface | | | contacting peptide. |
| | | exposed | | | Related N in |
| | | | | | siblings |
| H54HD | Reduce q, | H-CDR2 | −2.0 | No | Surface, but not |
| | germline | | | | contacting peptide. |
| | CDR | | | | Second shell |
| L285D | Reduce q | L-CDR1, | −1.0 | No | Surface, but not |
| | | Surface | | | contacting peptide. |
| | | exposed | | | Related N in |
| | | | | | siblings. Goes away |
| | | | | | from germline |
| L30SD | Reduce q, | L-CDR1, | −1.0 | Yes | Exposed, not |
| | Improve | exposed | | | contacting peptide. |
| | K_{D} | | | | Loses germline |
| L31RS | Reduce q, | L-CDR1 | −1.0 | Yes | Hbonds to L66oxy |
| | Germline | | | | and L52S, nearby |
| | & | | | | bound peptide and |
| | Improve | | | | may lock CDR |
| | K_{D} | | | | conformations |
| L565D | Reduce q | L-CDR2, | −1.0 | No | Surface, but not |
| | | Surface | | | contacting peptide. |
| | | exposed | | | R in sibling |
| H62DE | DS | H-CDR2 | 0.0 | No | Exposed loop. Salt |
| | Liability | | | | bridge with H65R. |
| | | | | | Goes away from |
| | | | | | germline |
| H62D5 | DS | H-CDR2 | +1.0 | No | Exposed loop. Salt |
| | Liability | | | | bridge with H65R. |
| | | | | | Goes away from |
| | | | | | germline |
| H63Sx² | DS | H-CDR2 | To be | No | Self interactions |
| | Liability | | determined | | |
| H99Dx | DG | H-CDR3 | To be | No | 3 self Hbonds and |
| | Liability | | determined | | nearly contacting |
| | | | | | bound peptide. |
| H100GA | DG | H-CDR3 | 0.0 | No | Backbone phi-psi |
| | Liability | | | | allows mutation but |
| | | | | | A may not fit |
| | | | | | against H98R- |
| | | | | | H107D salt bridge |
| L92NY | NS | L-CDR3, | 0.0 | Yes | Very near but not |
| | Liability | near but | | | contacting peptide |
| | & | not | | | |
| | Improve | contacting | | | |
| | K_{D} | peptide | | | |
| L93Sx | NS | L-CDR3, | To be | No | Very near but not |
| | Liability | near, but | determined | | contacting peptide. |
| | | not | | | S is in siblings. If |
| | | contacting | | | selecting this NS, |
| | | peptide | | | select L92N |
| | | | | | mutations |
| H31TN | Improve | H-CDR1 | 0.0 | Yes | Surface, but not |
| | K_{D} | | | | contacting peptide. |
| | | | | | Select N based on |
| | | | | | siblings, also select |
| | | | | | D to reduce q |
| H33A5 | Improve | H-CDR1 | 0.0 | Yes | Contacting peptide. |
| | K_{D} | | | | S may improve |
| | | | | | interactions. Goes |
| | | | | | away from germline |
| H53YN + | Improve | H-CDR2 | −1.0 | No | Y is barely |
| H55SE | K_{D} | | | | contacting peptide |
| | | | | | & very exposed. N |
| | | | | | may make Hbonds |
| | | | | | (in 1 potentially |
| | | | | | convergent Ab), but |
| | | | | | also make H55SE |
| | | | | | as E is in siblings, |
| | | | | | to avoid making an |
| | | | | | Nglyco site |
| H53YQ | Improve | H-CDR2 | 0.0 | No | Try Q to make |
| | K_{D} | | | | Hbonds (no concern |
| | | | | | about making an |
| | | | | | Nglyco site) |
| H56TR | Improve | H-CDR2 | +1.0 | Yes | Near but not |
| | K_{D} | | | | contacting peptide. |
| | | | | | R in siblings |
| H56TN | Improve | H-CDR2 | 0.0 | Yes | Near but not |
| | K_{D} | | | | contacting peptide. |
| | | | | | N in siblings |
| H57NQ | Improve | H-CDR2 | 0.0 | No | N makes good |
| | K_{D} | | | | Hbond to peptide |
| | | | | | backbone Oxy, but |
| | | | | | Q makes may make |
| | | | | | different, better |
| | | | | | Hbonds |
| H102SA | Improve | H-CDR3 | 0.0 | No | S in all sibs. |
| | K_{D} | | | | Contacts or weak |
| | | | | | Hbonds to 2 peptide |
| | | | | | side chains & Ab, |
| | | | | | but still solvent |
| | | | | | exposed |
| H102ST | Improve | H-CDR3 | 0.0 | No | Adding some |
| | K_{D} | | | | contact |
| H102SQ | Improve | H-CDR3 | 0.0 | No | May make |
| | K_{D} | | | | new/better Hbonds |
| | | | | | (N would create an |
| | | | | | Nglyco site |
| | | | | | w/H104S) |
| H102SH | Improve | H-CDR3 | 0.0 | No | May make |
| | K_{D} | | | | new/better Hbonds |
| H103ST | Improve | H-CDR3 | 0.0 | No | S in all sibs. Great |
| | K_{D} | | | | Hbond from peptide |
| | | | | | backbone NH. T |
| | | | | | may maintain |
| | | | | | Hbond but improve |
| | | | | | packing |
| H104ST | Improve | H-CDR3 | 0.0 | No | S in all sibs. Good |
| | K_{D} | | | | Hbond to peptide |
| | | | | | backbone Oxy. T |
| | | | | | may maintain |
| | | | | | Hbond but improve |
| | | | | | packing |
| H105FY | Improve | H-CDR3 | 0.0 | No | F in all sibs. Very |
| | K_{D} | | | | near peptide. Y |
| | | | | | may add Hbond to |
| | | | | | peptide backbone |
| | | | | | Oxy and/or from |
| | | | | | peptide N |
| L28SN | Improve | L-CDR1, | 0.0 | Yes | Exposed, not |
| | K_{D} | exposed | | | contacting peptide. |
| | | | | | Loses germline |
| L30SD | Improve | L-CDR1, | −1.0 | Yes | Exposed, not |
| | K_{D} & | exposed | | | contacting peptide. |
| | Reduce | | | | Loses germline |
| | q⁴ | | | | |
| L31RS | Improve | L-CDR1 | −1.0 | Yes | Hbonds to L66oxy |
| | K_{D} & | | | | and L52S, nearby |
| | Germline | | | | bound peptide and |
| | & | | | | mayd lock CDR |
| | Reduce q | | | | conformations |
| L49SA | Improve | L-FW2 | 0.0 | Yes | Hbond to peptide |
| | K_{D} | | | | |
| L49ST | Improve | L-FW2 | 0.0 | 0% | Hbond to peptide |
| | K_{D} | | | | |
| L50GK | Improve | L-CDR2, | +1.0 | Yes | Near peptide, space |
| | K_{D} & | near | | | for a side chain. K |
| | Germline | peptide | | | in most sibs |
| L50GQ | Improve | L-CDR2 | 0.0 | No | May make new |
| | K_{D} | | | | Hbonds to both |
| | | | | | peptide and Ab |
| L50GS | Improve | L-CDR2 | 0.0 | No | May add contact |
| | K_{D} | | | | and Hbond to |
| | | | | | peptide |
| L50GL | Improve | L-CDR2 | 0.0 | No | May add contacts |
| | K_{D} | | | | to peptide |
| L50GV | Improve | L-CDR2 | 0.0 | No | May add contacts |
| | K_{D} | | | | to peptide |
| L50GA | Improve | L-CDR2 | 0.0 | No | May add a little |
| | K_{D} | | | | contact to peptide |
| L53VS | Improve | L-CDR2 | 0.0 | Yes | Almost contacts |
| | K_{D} & | | | | aliphatic parts of |
| | Germline | | | | bound peptide, so |
| | | | | | polar S may reduce |
| | | | | | binding |
| L53VN | Improve | L-CDR2 | 0.0 | Yes | N found in siblings. |
| | K_{D} | | | | Near peptide but |
| | | | | | not predicted |
| | | | | | specific contacts |
| L94YH | Improve | L-CDR3 | +1.0 | No | H may make new |
| | K_{D} (& | | | | Hbond to peptide |
| | Reduce | | | | backbone Oxy. |
| | HΦ) | | | | Also, decreases |
| | | | | | small patch, HI |
| | | | | | from 2.94 to 2.66 |

¹“L32Wx” refers to the W on light chain residue 32 is mutated to an amino acid residue that is not W. Preferably, this residue is not an aromatic or hydrophobic amino acid as defined in [0035] of this application.
²“H63Sx” refers to the H residue on the heavy chain residue 63 is mutated to an amino acid residue that is not S. Preferably this residue is not a residue that re-creates a medium or low risk liability, i.e., “x” should not be G, A, D, or T (to avoid the dipeptides DG, DA, DD, DS, and DT; according to Table 4).
³Reduce HΦ refers to reduce antibody hydrophobicity, such as the reduction of the Hydrophobicity Index (HI; see Sharma 2014, supra) and/or the reduction of a surface hydrophobic patch.
⁴q refers to the net charge of the Fv of the antibody, see supra.

Illustrative antibodies comprising modifications to remove high liability residues and combinations thereof are shown in Table 10. Sequences are provided in Table 3.

TABLE 10
AB-000317 mutations and combinations thereof

| | | | | # | |
|---|---|---|---|---|---|
| Variant Name | Antibody ID | Purpose | Variant mutations | Mut | ΔCharge |
| GermLow_H23TA | | FW Germline | H23TA | 1 | 0.0 |
| GermLow_H40SA | | FW Germline | H40SA | 1 | 0.0 |
| GermLow_L20AT | | FW Germline | L20AT | 1 | 0.0 |
| GermLow | AB-007028 | FW Germline | H23TA + H40SA + L20AT | 3 | 0.0 |
| GermMed1 | AB-007029 | FW Germline | H2MV | 1 | 0.0 |
| GermMed23 | AB-007030 | FW Germline | H46QE + H61EA | 2 | 0.0 |
| GermMed4 | AB-007031 | FW Germline | L48MI | 1 | 0.0 |
| GermLowMed14 | AB-007032 | FW Germline | H2MV + H23TA + H40SA + | 5 | 0.0 |
| | | | L20AT + L48MI | | |
| GermLowMed23 | AB-007033 | FW Germline | H23TA + H40SA + H46QE + | 5 | 0.0 |
| | | | H61EA + L20AT | | |
| GermLowMed1234 | AB-007034 | FW Germline | H2MV + H23TA + H40SA + | 7 | 0.0 |
| | | | H46QE + H61EA + L20AT + | | |
| | | | L48MI | | |
| GermCDR_H31TS | AB-007035 | CDR Germline | H31TS | 1 | 0.0 |
| GermCDR_H54HD | AB-007036 | CDR Germline | H54HD | 1 | −2.0 |
| GermCDR_H65RK | AB-007037 | CDR Germline | H65RK | 1 | 0.0 |
| GermCDR_H108FY | AB-007038 | CDR Germline | H108FY | 1 | 0.0 |
| LiabHydro_L95FS | AB-007039 | Address | L95FS | 1 | 0.0 |
| | | hydrophobic | | | |
| | | patch liability | | | |
| LiabHydro_L95FY | AB-007040 | Address | L95FY | 1 | 0.0 |
| | | hydrophobic | | | |
| | | patch liability | | | |
| LiabCharge_H31TD | AB-007041 | Address net | H31TD | 1 | −1.0 |
| | | charge liability | | | |
| LiabCharge_L28SD | AB-007042 | Address net | L28SD | 1 | −1.0 |
| | | charge liability | | | |
| LiabCharge_L56SD | AB-007043 | Address net | L56SD | 1 | −1.0 |
| | | charge liability | | | |
| LiabDS_H62DE | AB-007044 | Address aspartate | H62DE | 1 | 0.0 |
| | | isomerization | | | |
| | | liability | | | |
| LiabDG_H100GA | AB-007045 | Address aspartate | H100GA | 1 | 0.0 |
| | | isomerization | | | |
| | | liability | | | |
| LiabNS_L92NY | AB-007046 | Address | L92NY | 1 | 0.0 |
| | | asparagine | | | |
| | | deamidation | | | |
| | | liability | | | |
| K_{D} _H31TN | AB-007047 | Address potency | H31TN | 1 | 0.0 |
| K_{D} _H33AS | AB-007048 | Address potency | H33AS | 1 | 0.0 |
| K_{D} _H53YN + | AB-007049 | Address potency | H53YN + H55SE | 2 | −1.0 |
| H55SE | | | | | |
| K_{D} _H53YQ | AB-007050 | Address potency | H53YQ | 1 | 0.0 |
| K_{D} _H56TR | AB-007051 | Address potency | H56TR | 1 | +1.0 |
| K_{D} _H56TN | AB-007052 | Address potency | H56TN | 1 | 0.0 |
| K_{D} _H57NQ | AB-007053 | Address potency | H57NQ | 1 | 0.0 |
| K_{D} _H102SA | AB-007054 | Address potency | H102SA | 1 | 0.0 |
| K_{D} _H102ST | AB-007055 | Address potency | H102ST | 1 | 0.0 |
| K_{D} _H102SQ | AB-007056 | Address potency | H102SQ | 1 | 0.0 |
| K_{D} _H102SH | AB-007057 | Address potency | H102SH | 1 | +1.0 |
| K_{D} _H103ST | AB-007058 | Address potency | H103ST | 1 | 0.0 |
| K_{D} _H104ST | AB-007059 | Address potency | H104ST | 1 | 0.0 |
| K_{D} _H105FY | AB-007060 | Address potency | H105FY | 1 | 0.0 |
| K_{D} _L30SD | AB-007061 | Address potency | L30SD | 1 | −1.0 |
| K_{D} _L31RS | AB-007062 | Address potency | L31RS | 1 | −1.0 |
| K_{D} _L49SA | AB-007063 | Address potency | L49SA | 1 | 0.0 |
| K_{D} _L49ST | AB-007064 | Address potency | L49ST | 1 | 0.0 |
| K_{D} _L50GK | AB-007065 | Address potency | L50GK | 1 | +1.0 |
| K_{D} _L50GQ | AB-007066 | Address potency | L50GQ | 1 | 0.0 |
| K_{D} _L50GS | AB-007067 | Address potency | L50GS | 1 | 0.0 |
| K_{D} _L50GL | AB-007068 | Address potency | L50GL | 1 | 0.0 |
| K_{D} _L50GV | AB-007069 | Address potency | L50GV | 1 | 0.0 |
| K_{D} _L50GA | AB-007070 | Address potency | L50GA | 1 | 0.0 |
| K_{D} _L53VS | AB-007071 | Address potency | L53VS | 1 | 0.0 |
| K_{D} _L53VN | AB-007072 | Address potency | L53VN | 1 | 0.0 |
| K_{D} _L94YH | AB-007073 | Address potency | L94YH | 1 | +1.0 |
| GermLow_L95FY | AB-007074 | Combo: Germline | H23TA + H40SA + L20AT + | 4 | 0.0 |
| | | framework & | 20AT + L9 [symbol] FY | | |
| | | address | | | |
| | | hydrophobic | | | |
| | | [symbol] + L | | | |

### Example 2. Methods

Forty-seven AB-000317 variants were evaluated for binding to the complete CSP protein and a series of linear peptides representing the immunodominant NANP repeat region. The goals of these antibodies were two-fold: 1) ameliorate potential liabilities predicted based on amino acid sequence while at least maintaining binding to the target and 2) improve the strength of antibody binding. Two assay platforms, bio-layer interferometry (BLI) and surface plasmon resonance (SPR), were used to quantify antibody-target binding strength. Five binding targets were evaluated in the SPR assay and six targets were evaluated in the BLI platform (Table 11).

TABLE 11
Target name sequence and assay in which the targets were
included

| | | Used in BLI, |
|---|---|---|
| | | SPR or both |
| Target Name | Peptide Sequence | assays |
| (NANP)6 | NANPNANPNANPNANPNANPNANP | Both |
| | (SEQ ID NO: 103) | |
| (NPNA)3 | NPNANPNANPNA | Both |
| | (SEQ ID NO: 104) | |
| (NVDP)3(NANP)2 | NVDPNANPNVDPNANPNVDP | Both |
| | (SEQ ID NO: 105) | |
| NANPNVDPNANP | NPDPNANPNVDPNANP | Both |
| | (SEQ ID NO: 106) | |
| NANPNVDP | DPNANPNVDPNA | BLI only |
| | (SEQ ID NO: 107) | |
| N-Interface | KQPADGNPDPNANPN | BLI only |
| | (SEQ ID NO: 108) | |
| CSP Protein | | SPR only |

For BLI, each of the targets specified in Table 11 was biotinylated and immobilized to streptavidin sensors. Each antibody was evaluated in duplicate at 5 μg/mL. If variation between the two duplicates was >3-fold, the antibody-target measurement was repeated.

For SPR, each antibody was either directly coupled to a Carterra Chip or coupled using a goat anti-human Fc antibody. The uncoupled antibodies were washed off and various concentration gradients of the targets were flowed over the antibodies, where the highest concentration of each target was in the range 0.5-8 μg/mL. Each antibody was immobilized in two different locations on the chip to allow for duplicate measurements. The affinity for each antibody-target combination was determined using 4-5 target concentrations in Mathematica software. If variation between the two duplicates was >3-fold, the antibody-target measurement was repeated.

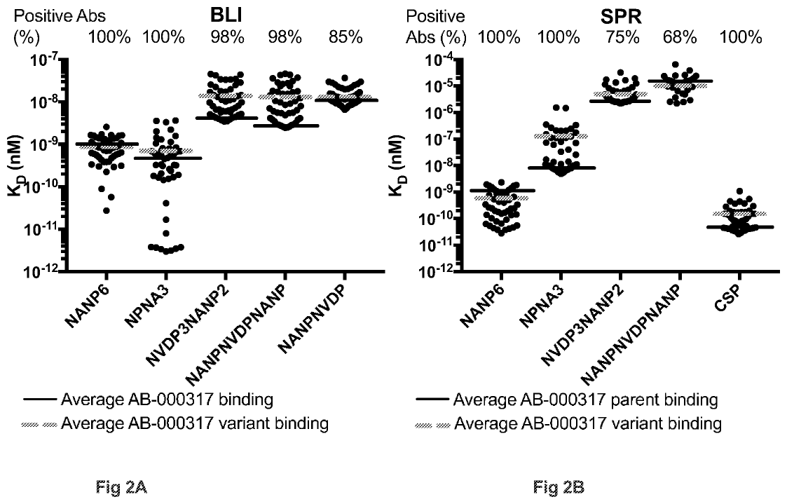

While the data generated by the BLI and SPR assays are similar, the assays were designed with opposite orientations of the target and antibody. Specifically, the target was immobilized while the antibody flowed over it in the BLI assay, while the SPR assay was designed so that the antibody was immobilized and the target flowed over it. Given these orientations, an antibody, when evaluated in the BLI assay, would be more likely to engage in binding interactions that involve multiple target molecules. As such, the binding of antibodies to targets in the BLI assay may exhibit more similarities to binding the complete CSP protein, which coats the surface of the malaria sporozoite. In contrast, the activity measured in the SPR assay would more accurately represent an interaction between an antibody F(ab) and a single target molecule. Oyen et al (2017) determined that the minimum epitope required for AB-000317 was 2 NANP repeats, and at least 2.5 repeats were required for stronger binding. While AB-000317 and its variants bound within 3-fold of the average binding of AB-000317 to the (NVDP)3(NANP2), NANPNVDPNANP, NANPNVDP and NVDPNANP peptides (FIG. 2A-2B) in both BLI and SPR assays, we restricted our analysis of the antibody variants to targets that included the minimum epitope required for strong binding: (NANP)6, (NPNA)3 and CSP.

Summary of Antibody Variants that Evaluate Germlined Residues:

Eleven variants (AB-007028, AB-007029, AB-007030, AB-007031, AB-007032, AB-007033, AB-007034, AB-007035, AB-007036, AB-007037, AB-007038) were designed to germline antibodies by mutating residues in either the framework regions or CDRs to reduce the risk of antibody-directed immunogenicity. Seven ((AB-007028, AB-007029, AB-007030, AB-007031, AB-007032, AB-007033, AB-007034) variants evaluated seven mutations in the antibody framework regions either individually or in combination, and four variants (AB-007035, AB-007036, AB-007037, AB-007038) evaluated four individual mutations at separate residues located within the antibody CDRs.

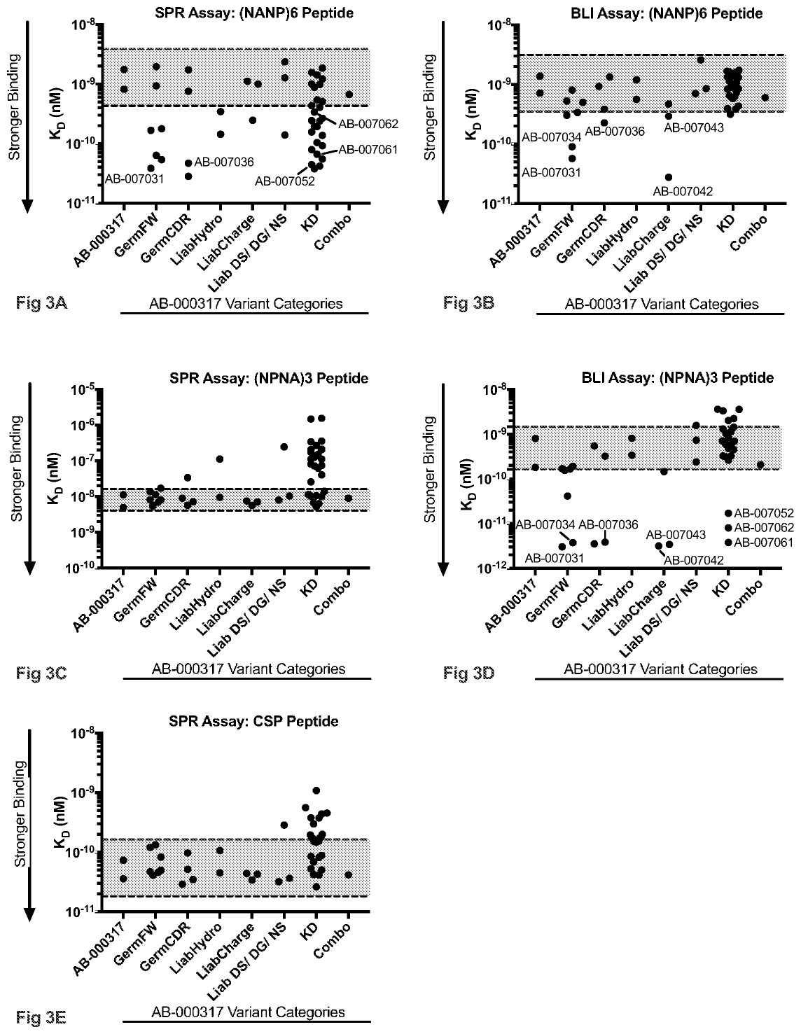

All 11 antibodies either maintained binding comparable to or improved as compared to AB-000317. Three of the eleven antibodies demonstrate potentially interesting activity. AB-007031-1 bound more tightly than AB-000317 in three target-assay combinations: (NANP)6-SPR, (NANP)6-BLI and (NPNA)3-BLI, and maintained binding affinity comparable to AB-000317 in the other two target-assays. AB-007034-1, which incorporates all of the suggested framework modifications into one variant, exhibited improved binding in both BLI assays and maintained binding comparable to AB-000317 in the three SPR assays. Lastly, AB-0007036 demonstrated superior binding as compared to AB-000317 in three target-assay combinations: (NANP)6-SPR, (NANP)6-BLI and (NPNA)3-BLI (FIG. 3A-3E, Table 12A and Table 12B). The first column shows information of the antibody IDs and their respective batch numbers of the production. “GermFW” are variants in which at least one residue in the framework have been mutated to the corresponding residue in the germline sequence. “GermCDR” are variants in which at least one residue in the CDRs have been mutated to the corresponding residue in the germline sequence. “K_{D}”, as used in the Variant modification and Variant name columns of Table 12,are variants in which at least one residue has been mutated to improve binding strength,

TABLE 12A
In vitro binding

| Antibody | | Variant | Variant | NANP6 | NANP6 | NPNA3 |
|---|---|---|---|---|---|---|
| ID-Batch No. | Purpose | name | mutations | K_{D} BLI | K_{D} SPR | K_{D} BLI |
| AB-000317-2 | Parent | | | 7.14E−10 | 1.75E−09 | 1.80E−10 |
| AB-000317-3 | Parent | | | 1.38E−09 | 8.17E−10 | 8.00E−10 |
| AB-007028-1 | GermFW | GermLow | H23TA + H40SA + | 5.01E−10 | 1.66E−10 | 4.13E−11 |
| | | | L20AT | | | |
| AB-007029-1 | GermFW | GermMed1 | H2MV | 8.03E−10 | 1.77E−10 | 1.66E−10 |
| AB-007030-1 | GermFW | GermMed23 | H46QE + H61EA | 5.26E−10 | 5.37E−11 | 1.92E−10 |
| AB-007031-1 | GermFW | GermMed4 | L48MI | 5.69E−11 | 3.84E−11 | 3.00E−12 |
| AB-007032-1 | GermFW | GermLowMed14 | H2MV + H23TA + | 3.38E−10 | 1.96E−09 | 1.69E−10 |
| | | | H40SA + L20AT + | | | |
| | | | L48MI | | | |
| AB-007033-1 | GermFW | GermLowMed23 | H23TA + H40SA + | 3.03E−10 | 6.31E−11 | 1.54E−10 |
| | | | H46QE + H61EA + | | | |
| | | | L20AT | | | |
| AB-007034-1 | GermFW | GermLowMed1234 | H2MV + H23TA + | 8.99E−11 | 9.32E−10 | 3.72E−12 |
| | | | H40SA + H46QE + | | | |
| | | | H61EA + L20AT + | | | |
| | | | L48MI | | | |
| AB-007035-1 | GermCDR | GermCDR_H31TS | H31TS | 9.27E−10 | 7.57E−10 | 3.22E−10 |
| AB-007036-1 | GermCDR | GermCDR_H54HD | H54HD | 2.26E−10 | 4.66E−11 | 3.82E−12 |
| AB-007037-1 | GermCDR | GermCDR_H65RK | H65RK | 3.84E−10 | 1.73E−09 | 3.50E−12 |
| AB-007038-1 | GermCDR | GermCDR_H108FY | H108FY | 1.34E−09 | 2.81E−11 | 5.44E−10 |
| AB-007039-1 | LiabHydro | LiabHydro_L95FS | L95FS | 1.19E−09 | 1.43E−10 | 8.12E−10 |
| AB-007040-1 | LiabHydro | LiabHydro_L95FY | L95FY | 5.60E−10 | 3.44E−10 | 3.37E−10 |
| AB-007041-1 | LiabCharge | LiabCharge_H31TD | H31TD | 4.70E−10 | 2.46E−10 | 1.45E−10 |
| AB-007042-1 | LiabCharge | LiabCharge_L28SD | L285D | 2.76E−11 | 9.98E−10 | 3.14E−12 |
| AB-007043-1 | LiabCharge | LiabCharge_L56SD | L565D | 2.93E−10 | 1.11E−09 | 3.41E−12 |
| AB-007044-1 | LiabDS | LiabDS_H62DE | H62DE | 8.50E−10 | 1.39E−10 | 2.39E−10 |
| AB-007045-1 | LiabDG | LiabDG_H100GA | H100GA | 7.03E−10 | 2.35E−09 | 7.34E−10 |
| AB-007046-1 | LiabNS | LiabNS_L92NY | L92NY | 2.58E−09 | 1.27E−09 | 1.57E−09 |
| AB-007047-1 | K_{D} | K_{D}_H31TN | H31TN | 1.32E−09 | 1.56E−10 | 5.67E−10 |
| AB-007048-1 | K_{D} | K_{D} _H33AS | H33AS | 1.46E−09 | 1.00E−09 | 3.33E−09 |
| AB-007049-1 | K_{D} | K_{D}_H53YN + H55SE | H53YN + H55SE | 5.76E−10 | 9.20E−11 | 3.26E−10 |
| AB-007050-1 | K_{D} | K_{D}_H53YQ | H53YQ | 8.56E−10 | 5.40E−10 | 1.05E−09 |
| AB-007051-1 | K_{D} | K_{D}_H56TR | H56TR | 1.08E−09 | 5.07E−10 | 5.91E−10 |
| AB-007052-1 | K_{D} | K_{D}_H56TN | H56TN | 3.92E−10 | 4.42E−11 | 1.69E−11 |
| AB-007053-1 | K_{D} | K_{D}_H57NQ | H57NQ | 1.37E−09 | 1.42E−09 | 5.52E−10 |
| AB-007054-1 | K_{D} | K_{D}_H102SA | H102SA | 1.72E−09 | 5.52E−11 | 6.10E−10 |
| AB-007055-1 | K_{D} | K_{D}_H102ST | H102ST | 1.61E−09 | 4.03E−10 | 3.62E−09 |
| AB-007056-1 | K_{D} | K_{D}_H102SQ | H102SQ | 8.80E−10 | 1.04E−10 | 2.63E−10 |
| AB-007057-1 | K_{D} | K_{D}_H102SH | H102SH | 9.76E−10 | 4.19E−11 | 3.11E−10 |
| AB-007058-1 | K_{D} | K_{D}_H103ST | H103ST | 1.14E−09 | 1.37E−10 | 1.30E−09 |
| AB-007059-1 | K_{D} | K_{D}_H104ST | H104ST | 1.41E−09 | 2.66E−10 | 4.78E−10 |
| AB-007060-1 | K_{D} | K_{D}_H105FY | H105FY | 8.98E−10 | 1.21E−09 | 1.14E−09 |
| AB-007061-1 | K_{D} | K_{D}_L30SD | L30SD | 3.85E−10 | 6.57E−11 | 3.76E−12 |
| AB-007062-1 | K_{D} | K_{D}_L31RS | L31RS | 6.07E−10 | 3.38E−10 | 7.96E−12 |
| AB-007063-1 | K_{D} | K_{D}_L49SA | L495A | 1.64E−09 | 1.94E−10 | 6.91E−10 |
| AB-007064-1 | K_{D} | K_{D}_L49ST | L495T | 4.34E−10 | 1.92E−10 | 4.53E−10 |
| AB-007065-1 | K_{D} | K_{D}_L50GK | L50GK | 7.29E−10 | 2.38E−10 | 2.23E−09 |
| AB-007066-1 | K_{D} | K_{D}_L50GQ | L50GQ | 3.15E−10 | 9.79E−10 | 3.58E−09 |
| AB-007067-1 | K_{D} | K_{D}_L50GS | L50GS | 6.47E−10 | 2.44E−10 | 2.02E−09 |
| AB-007068-1 | K_{D} | K_{D}_L50GL | L50GL | 1.59E−09 | 1.56E−09 | 7.03E−10 |
| AB-007069-1 | K_{D} | K_{D}_L50GV | L50GV | 1.33E−09 | 1.85E−09 | 1.43E−09 |
| AB-007070-1 | K_{D} | K_{D}_L50GA | L50GA | 8.34E−10 | 7.86E−11 | 6.80E−10 |
| AB-007071-1 | K_{D} | K_{D}_L53VS | L53VS | 1.66E−09 | 4.33E−10 | 6.94E−10 |
| AB-007072-1 | K_{D} | K_{D}_L53VN | L53VN | 1.65E−09 | 3.76E−11 | 8.42E−10 |
| AB-007073-1 | K_{D} | K_{D}_L94YH | L94YH | 6.27E−10 | 8.71E−10 | 3.26E−10 |
| AB-007074-1 | GermFW + | GermLow_L95FY | H23TA + H40SA + | 6.01E−10 | 6.65E−10 | 2.06E−10 |
| | LiabHydro | | L20AT + L95FY | | | |
| AB-000329-2 | Negative | N/A | N/A | No binding | No binding | No binding |
| | control | | | | | |
| | #1 | | | | | |
| AB-003853-7 | Negative | N/A | N/A | No binding | No binding | No binding |
| | control | | | | | |
| | #2 | | | | | |

TABLE 12B
In vitro binding (cont'd)

| Antibody | NPNA3 | NVDP3NANP2 | NVDP3NANP2 | NANPNVDPNANP | NANPNVDPNANP | NANPNVDP | CSP K_{D} |
|---|---|---|---|---|---|---|---|
| ID-Batch No. | K_{D} SPR | K_{D} BLI | K_{D} SPR | K_{D} BLI | K_{D} SPR | K_{D} BLI | (SPR) |
| AB-000317-2 | 4.91E−09 | 4.27E−09 | 2.59E−06 | 2.76E−09 | 1.29E−05 | 9.20E−09 | 3.58E−11 |
| AB-000317-3 | 1.12E−08 | 3.73E−09 | 2.91E−06 | 2.51E−09 | 1.58E−05 | 1.27E−08 | 7.30E−11 |
| AB-007028-1 | 5.37E−09 | 4.26E−09 | 2.35E−06 | 2.93E−09 | 1.59E−05 | 9.18E−09 | 4.57E−11 |
| AB-007029-1 | 1.13E−08 | 6.67E−09 | 4.79E−06 | 5.62E−09 | 2.62E−05 | 1.32E−08 | 1.20E−10 |
| AB-007030-1 | 6.88E−09 | 4.03E−09 | 2.57E−06 | 2.84E−09 | 6.52E−06 | 7.92E−09 | 1.32E−10 |
| AB-007031-1 | 8.02E−09 | 3.50E−09 | 2.59E−06 | 2.89E−09 | 5.21E−06 | 7.02E−09 | 4.96E−11 |
| AB-007032-1 | 1.36E−08 | 6.68E−09 | 6.54E−06 | 6.42E−09 | 4.83E−06 | 1.39E−08 | 4.69E−11 |
| AB-007033-1 | 8.02E−09 | 3.47E−09 | 2.68E−06 | 2.62E−09 | 2.58E−06 | 8.08E−09 | 4.08E−11 |
| AB-007034-1 | 1.70E−08 | 6.14E−09 | 6.71E−06 | 5.44E−09 | 2.41E−05 | 1.15E−08 | 8.24E−11 |
| AB-007035-1 | 5.73E−09 | 5.09E−09 | 2.69E−06 | 2.61E−09 | 9.75E−06 | 9.28E−09 | 5.13E−11 |
| AB-007036-1 | 3.35E−08 | 1.74E−08 | 1.67E−05 | 1.69E−08 | 1.75E−05 | 2.96E−08 | 9.67E−11 |
| AB-007037-1 | 7.14E−09 | 4.44E−09 | 2.69E−06 | 2.47E−09 | 2.24E−06 | 8.41E−09 | 3.46E−11 |
| AB-007038-1 | 8.97E−09 | 5.82E−09 | 3.06E−06 | 3.81E−09 | 1.31E−05 | 1.09E−08 | 2.90E−11 |
| AB-007039-1 | 1.12E−07 | 7.26E−09 | 8.25E−06 | 7.97E−09 | 1.47E−05 | 1.53E−08 | 1.06E−10 |
| AB-007040-1 | 9.50E−09 | 4.67E−09 | 3.11E−06 | 4.17E−09 | 3.73E−06 | 9.56E−09 | 4.47E−11 |
| AB-007041-1 | 5.64E−09 | 6.65E−09 | 3.93E−06 | 5.57E−09 | 2.52E−06 | 1.21E−08 | 4.39E−11 |
| AB-007042-1 | 7.04E−09 | 3.90E−09 | 2.31E−06 | 2.99E−09 | 1.17E−05 | 8.56E−09 | 3.40E−11 |
| AB-007043-1 | 7.51E−09 | 5.20E−09 | 2.59E−06 | 4.87E−09 | 1.63E−05 | 1.04E−08 | 4.26E−11 |
| AB-007044-1 | 7.96E−09 | 5.99E−09 | 2.24E−06 | 3.47E−09 | 1.27E−05 | 8.61E−09 | 3.19E−11 |
| AB-007045-1 | 2.46E−07 | 4.39E−08 | 0.00E+00 | 4.36E−08 | 0.00E+00 | 2.82E−08 | 2.84E−10 |
| AB-007046-1 | 1.03E−08 | 1.16E−08 | 2.81E−06 | 9.15E−09 | 1.16E−05 | 1.11E−08 | 3.64E−11 |
| AB-007047-1 | 1.14E−08 | 8.11E−09 | 3.68E−06 | 5.43E−09 | 9.37E−06 | 1.00E−08 | 1.75E−10 |
| AB-007048-1 | 1.55E−06 | 3.46E−08 | 0.00E+00 | 2.86E−08 | 0.00E+00 | 0.00E+00 | 4.37E−10 |
| AB-007049-1 | 2.58E−08 | 1.30E−08 | 7.14E−06 | 1.23E−08 | 2.95E−06 | 2.12E−08 | 1.52E−10 |
| AB-007050-1 | 2.71E−07 | 3.34E−08 | 0.00E+00 | 3.74E−08 | 0.00E+00 | 0.00E+00 | 1.47E−10 |
| AB-007051-1 | 5.19E−09 | 4.89E−09 | 2.49E−06 | 3.23E−09 | 8.84E−06 | 6.57E−09 | 4.14E−11 |
| AB-007052-1 | 6.31E−09 | 6.28E−09 | 2.56E−06 | 3.83E−09 | 7.14E−06 | 7.34E−09 | 2.61E−11 |
| AB-007053-1 | 1.12E−07 | 2.58E−08 | 0.00E+00 | 2.35E−08 | 0.00E+00 | 2.47E−08 | 2.98E−10 |
| AB-007054-1 | 1.05E−08 | 1.02E−08 | 5.67E−06 | 8.50E−09 | 1.76E−05 | 1.37E−08 | 1.70E−10 |
| AB-007055-1 | 1.47E−06 | 0.00E+00 | 0.00E+00 | 0.00E+00 | 0.00E+00 | 0.00E+00 | 3.74E−10 |
| AB-007056-1 | 6.20E−08 | 2.49E−08 | 0.00E+00 | 2.36E−08 | 0.00E+00 | 2.96E−08 | 4.20E−11 |
| AB-007057-1 | 4.03E−08 | 1.12E−08 | 9.09E−06 | 1.05E−08 | 2.01E−05 | 1.53E−08 | 1.53E−10 |
| AB-007058-1 | 3.39E−07 | 4.22E−08 | 0.00E+00 | 4.63E−08 | 0.00E+00 | 0.00E+00 | 1.49E−10 |
| AB-007059-1 | 1.74E−07 | 1.98E−08 | 1.75E−05 | 1.74E−08 | 0.00E+00 | 2.58E−08 | 1.08E−09 |
| AB-007060-1 | 2.12E−07 | 4.52E−08 | 0.00E+00 | 4.32E−08 | 0.00E+00 | 0.00E+00 | 1.99E−10 |
| AB-007061-1 | 6.69E−09 | 5.68E−09 | 4.00E−06 | 4.11E−09 | 1.46E−05 | 1.23E−08 | 5.04E−11 |
| AB-007062-1 | 1.00E−08 | 1.36E−08 | 6.25E−06 | 1.43E−08 | 2.50E−05 | 2.24E−08 | 8.49E−11 |
| AB-007063-1 | 1.41E−08 | 9.73E−09 | 3.89E−06 | 7.03E−09 | 1.22E−05 | 1.31E−08 | 5.24E−11 |
| AB-007064-1 | 1.52E−07 | 3.37E−08 | 0.00E+00 | 3.09E−08 | 0.00E+00 | 3.67E−08 | 5.56E−10 |
| AB-007065-1 | 1.25E−07 | 9.71E−09 | 1.26E−05 | 9.73E−09 | 0.00E+00 | 1.58E−08 | 4.54E−10 |
| AB-007066-1 | 3.57E−07 | 2.24E−08 | 0.00E+00 | 2.66E−08 | 0.00E+00 | 0.00E+00 | 3.76E−10 |
| AB-007067-1 | 2.07E−07 | 3.40E−08 | 0.00E+00 | 3.61E−08 | 0.00E+00 | 0.00E+00 | 8.77E−11 |
| AB-007068-1 | 1.11E−07 | 1.71E−08 | 3.24E−05 | 1.83E−08 | 0.00E+00 | 2.18E−08 | 1.52E−10 |
| AB-007069-1 | 2.04E−07 | 2.87E−08 | 0.00E+00 | 2.75E−08 | 0.00E+00 | 2.66E−08 | 1.74E−10 |
| AB-007070-1 | 8.03E−08 | 1.44E−08 | 1.16E−05 | 1.55E−08 | 6.53E−05 | 1.98E−08 | 1.77E−10 |
| AB-007071-1 | 1.02E−08 | 1.10E−08 | 4.87E−06 | 1.04E−08 | 2.38E−05 | 1.29E−08 | 6.83E−11 |
| AB-007072-1 | 7.39E−08 | 1.45E−08 | 1.38E−05 | 1.55E−08 | 3.87E−05 | 1.69E−08 | 1.94E−10 |
| AB-007073-1 | 7.20E−08 | 1.81E−08 | 1.95E−05 | 1.79E−08 | 1.65E−05 | 2.03E−08 | 8.16E−11 |
| AB-007074-1 | 8.94E−09 | 7.96E−09 | 3.92E−06 | 6.05E−09 | 1.28E−05 | 1.02E−08 | 4.15E−11 |
| AB-000329-2 | No binding | No binding | No binding | No binding | No binding | No binding | No binding |
| AB-003853-7 | No binding | No binding | No binding | No binding | No binding | No binding | No binding |

These binding data suggest that all of the germline and CDR modifications designed to minimize a predicted liability maintain binding that is at least as strong as parent antibody AB-000317. As such, new antibody variants could include all 11 of the modifications without detrimentally impacting binding.

### Summary of Antibody Variants Made to Eliminate Predicted Biophysical Liabilities

Eight antibodies (AB-007039, AB-007040, AB-007041, AB-007042, AB-007043, AB-007044, AB-007045, AB-007046) evaluated individual mutations designed to address potential liabilities predicted on the basis of sequence that could affect the stability or other biophysical properties of the antibodies. These included a 1) hydrophobic patch liability (LiabHydro); 2) net charge liability (LiabCharge); 3) aspartate isomerization liability (LiabDS) and 4) asparagine deamidation liability (LiabNS). All eight of the antibodies demonstrated comparable or superior (defined as ≥3-fold lower) binding as compared to AB-000317 in all five target-assay combinations with only one exception. Two antibodies, AB-007042 and AB-007043, demonstrate interesting activity. Both of these antibodies bind more strongly to the (NANP)6 and (NPNA)3 peptides by BLI than AB-000317 and comparably to both peptides and CSP in the SPR assay (FIG. 3A-3E, Table 12A and 12B).

These binding data suggest that the antibodies designed to minimize predicted biophysical liabilities maintain binding that is at least as strong as, and in some cases stronger than, parent antibody AB-000317. As such, new antibody variants that include combinations of these mutations should be tested to further improve antibody stability and reduce developability risks.

### Summary of Antibodies Designed to Improve Antibody Binding Strength

Twenty-seven antibodies (AB-007047, AB-007048, AB-007049, AB-007050, AB-007051, AB-007052, AB-007053, AB-007054, AB-007055, AB-007056, AB-007057, AB-007058, AB-007059, AB-007060, AB-007061, AB-007062, AB-007063, AB-007064, AB-007065, AB-007066, AB-007067, AB-007068, AB-007069, AB-007070, AB-007071, AB-007072, AB-007073) were designed to strengthen parent AB-000317 binding by testing single mutations at 16 different residues. For some residues like H102 and L50, we designed multiple antibodies each testing a different mutation while for other residues, we only evaluated one mutation.

When evaluated for binding against (NANP)6, all 27 antibodies demonstrated comparable or improved (defined as ≥3-fold better than AB-000317) binding in both the SPR and BLI assays. When the antibody variants were evaluated against (NPNA)3, 10 demonstrated binding comparable to AB-000317 in the SPR assay, and 22 demonstrated comparable or improved binding in the BLI assay. Fourteen antibodies maintained binding comparable to AB-000317 when evaluated in the CSP-SPR assay combination. Three antibodies, AB-007053-1, AB-007061-1 and AB-007062-1 demonstrated at least 1-log fold improvement as compared to AB-000317 binding in the (NANP)6-SPR and (NPNA)3-BLI assay-target combinations and comparable binding to AB-000317 in the other three target-assays (FIG. 3A-3E, Table 12A and 12B). AB-007053 includes a mutation at position 56 of the heavy chain, while antibodies AB-007061 and AB-007062 include mutations at positions 30 and 31 of the light chain, respectively. These three residues may be further exploited to identify additional mutations that improve target binding.

One antibody variant, AB-007074, was designed to evaluate multiple mutations combined into one antibody. This variant includes three mutations to revert residues back to germline in the framework region of AB-000317. A 4th mutation is designed to address the hydrophobic patch in the light chain (position 95). Consistent with the binding data for the two antibodies evaluating these mutations independently (AB-007028 evaluated the three germline mutations and AB-007040 evaluated the mutation to eliminate the hydrophobic patch), AB-007074 demonstrated comparable binding to parent antibody AB-000317 in all five target-assay combinations (FIG. 3A-3E, Table 12A and 12B). This shows that the variants tested can eliminate predicted liabilities while maintaining or improving antibody binding. We predict that other variant antibodies could be generated combining additional mutations thus leading to further improvements.

### Example 3 In Vivo Performance of the Antibody Variants

Five antibodies, AB-007031, AB-007033, AB-007040, AB-007041 and AB-007042 were also evaluated for in vivo activity in a mouse malaria liver burden assay, as described in Flores-Garcia Y, et al. Optimization of an in vivo model to study immunity to Plasmodium falciparum pre-erythrocytic stages. Malar J. 2019; 18(1):426, doi:10.1186/s12936-019-3055-9. For each antibody, five C57Bl/6 mice per experimental or control arm were administered 100 μg of antibody 16 hours prior to intravenous infection with fluorescent P. berghei sporozoites. Forty-two hours following parasite challenge, the sporozoite liver load was quantified by bioluminescence. For each experimental mouse, the percent liver burden was calculated by subtracting the average background luminescence measured from two un-treated, naïve mice and calculating the percent reduction as compared to the average luminescence measured in five un-treated, infected mice. The average percent reduction was reported for each of the experimental antibody groups.

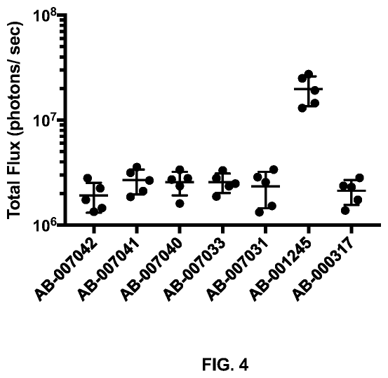

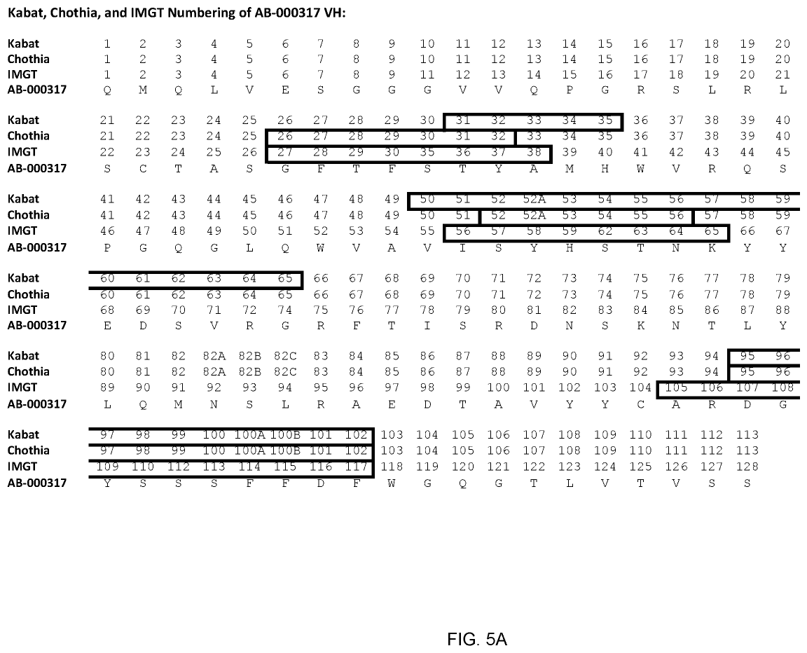

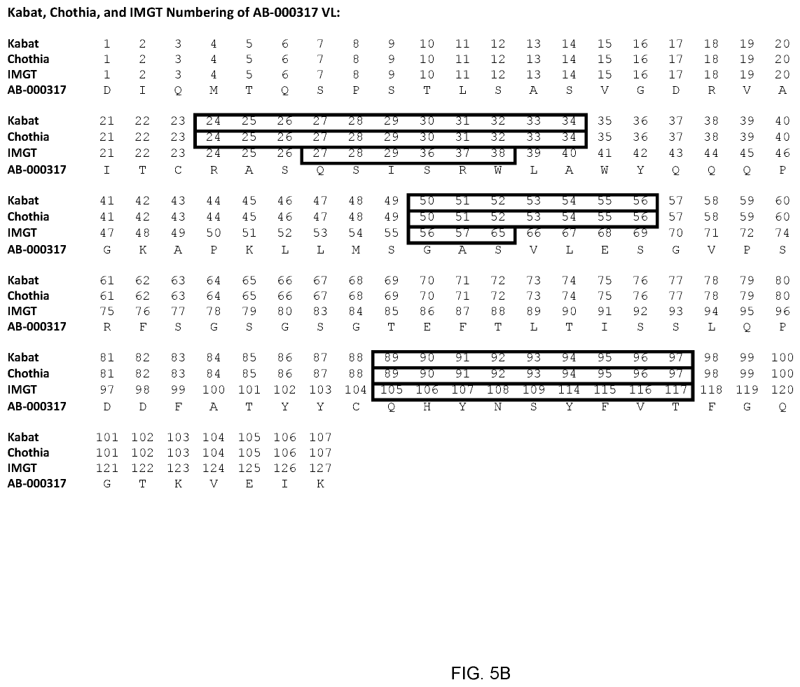

All five of the antibodies exhibited binding comparable to, or in some assay-target combinations, superior to AB-000317. Consistent with the binding data, all 5 antibodies also reduced the percent liver burden loads in malaria-infected mice significantly better than the negative control AB-001245 (p=0.008, Mann-Whitney test) and similar to the parent antibody AB-000317 (p>0.05, Mann-Whitney test; Table 13 and FIG. 4). These data demonstrate that antibody variants that mutate either a single residue or a combination of residues can both eliminate potential liabilities while maintaining in vivo activity.

TABLE 13
The five AB-000317 variants evaluated in vivo exhibit
similar activity to the parent antibody

| | | Average reduction in |
|---|---|---|
| Antibody ID | Variant purpose & mutation | liver burden (%) |
| AB-007031 | FW Germline: L48MI | 88.0% |
| AB-007033 | FW Germline: | 86.4% |
| | H23TA + H40SA + H46QE + | |
| | H61EA + L20AT | |
| AB-007040 | Hydrophobic patch liability: | 86.5% |
| | LiabHydro_L95FY | |
| AB-007041 | Net charge liability | 85.9% |
| | LiabCharge_H31TD | |
| AB-007042 | Net charge liability: | 89.7% |
| | LiabCharge_L28SD | |
| AB-000317 | Parent antibody | 88.7% |
| AB-001245 | Negative control antibody | 2.95% |

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference for the purposes in the context of which they are cited.

1. An anti-circumsporozoite (CSP) antibody comprising a heavy chain variable (V_{H}) region and a light chain variable (V_{L}) region, wherein:
(a) the V_{H} region comprises at least one substitution in a CDR1 sequence comprising ²⁶GFTFSTYAMH³⁵, a CDR2 sequence comprising ⁵⁰VISYHSTNKYYEDSVRG⁶⁶, and a CDR3 sequence comprising ⁹⁷ARDGYSSSFFDF¹⁰⁸ as numbered with reference to SEQ ID NO:1; wherein the at least one substitution is selected from the group consisting of D at position 30; S, D, or N at position 31; S at position 33; N or Q at position 53; D at position 54, G or E at position 55; S, R, or N at position 56; Q at position 57; A at position 61; E or S at position 62; K at position 65; A at position 100; A, T, Q, or H at position 102; T at position 103, T at position 104, Y at position 105; and Y at position 108; and
the V_{L} region comprises: (i) a CDR1 sequence comprising ²⁴RASQSISRWLA³⁴, a CDR2 sequence comprising ⁵⁰GASVLES⁵⁶, and a CDR3 sequence comprising ⁸⁹QHYNSYFVT⁹⁷ as numbered with reference to SEQ ID NO:2; or (ii) at least one substitution in the CDR1 sequence, CDR2 sequence, or CDR3 sequence, wherein the at least one substitution is selected from the group consisting of D or N at position 28; D at position 30; S at position 31; K, Q, S, L, V, or A at position 50; S or N at position 53; D at position 56; Q at position 90, Y at position 92; H at position 94, S or Y at position 95; and W at position 96; or
(b) the V_{H} region comprises: (i) a CDR1 sequence comprising ²⁶GFTFSTYAMH³⁵, a CDR2 sequence comprising ⁵⁰VISYHSTNKYYEDSVRG⁶⁶, and a CDR3 sequence comprising ⁹⁷ARDGYSSSFFDF¹⁰⁸ as numbered with reference to SEQ ID NO:1; or (ii) at least one substitution in the CDR1 sequence, the CDR2 sequence, or the CDR3 sequence, wherein the at least one substitution is selected from the group consisting of D at position 30; S, D, or N at position 31; S at position 33; N or Q at position 53; D at position 54, G or E at position 55; S, R, or N at position 56; Q at position 57; A at position 61; E or S at position 62; K at position 65; A at position 100; A, T, Q, or H at position 102; T at position 103, T at position 104, Y at position 105; and Y at position 108; and
the V_{L} region comprises at least one substitution in a CDR1 sequence comprising ²⁴RASQSISRWLA³⁴, a CDR2 sequence comprising ⁵⁰GASVLES⁵⁶, and a CDR3 sequence comprising ⁸⁹QHYNSYFVT⁹⁷ as numbered with reference to SEQ ID NO:2; wherein the at least one substitution is selected from the group consisting of D or N at position 28; D at position 30; S at position 31; K, Q, S, L, V, or A at position 50; S or N at position 53; D at position 56; Q at position 90, Y at position 92; H at position 94, S or Y at position 95; and W at position 96.

2. (canceled)

3. The anti-CSP antibody of claim 1, wherein the V_{H} region comprises at least one of the following, as numbered with reference to SEQ ID NO: 1: V at position 2, A or E at position 23, A at position 40, K at position 43, or E at position 46.

4. The anti-CSP antibody of claim 1, wherein the V_{L} region comprises at least one of the following, as numbered with reference to SEQ ID NO:2: T at position 20; K at position 39; I at position 48; or A, T, or Y at position 49.

5. The anti-CSP antibody of claim 1, wherein the V_{H} region has at least 70% identity to SEQ ID NO:1; and the V_{L} region has at least 70% identity to SEQ ID NO:2.

6. The anti-CSP antibody of claim 1, wherein the V_{H} region has at least 80% identity to SEQ ID NO:1; and/or the V_{L} region has at least 80% identity to SEQ ID NO:2.

7. The anti-CSP antibody of claim 1, wherein the V_{H} region has at least 90% identity to SEQ ID NO:1; and/or the V_{L} region has at least 90% identity to SEQ ID NO:2.

8. The anti-CSP antibody of claim 1, wherein the V_{H} region has at least 95% identity to SEQ ID NO:1; and/or the V_{L} region has at least 95% identity to SEQ ID NO:2.

9. An anti-CSP antibody comprising a heavy chain variable (V_{H}) region and a light chain variable (V_{L}) region, wherein:
(a) the V_{H} region has at least 70% identity to SEQ ID NO:1; and comprises a CDR1 as set forth in Table 1 or the CDR1 of Table 1 in which 1, 2, or 3 amino acids are substituted, a CDR2 as set forth in Table 1 or the CDR2 of Table 1 in which 1, 2, 3, 4, 5, 6, 7, 8, or 9 amino acids are substituted, and a CDR3 as set forth in Table 1 or the CDR3 of Table 1 in which 1, 2, 3, 4, 5, 6, or 7 amino acids are substituted; and at least one of the following substitutions as determined with reference to SEQ ID NO:1: V at position 2, A or E at position 23, A at position 40, K at position 43, or E at position 46; and
the V_{L} region (i) comprises the amino acid sequence of SEQ ID NO:2; or (ii) comprises a CDR1 as set forth in Table 1 or the CDR1 of Table 1 in which 1, 2, 3, or 4 amino acids are substituted, a CDR2 as set forth in Table 1 or the CDR2 of Table 1 in which 1, 2, or 3 amino acids are substituted, and a CDR3 as set forth in Table 1 or the CDR3 of Table 1 in which 1, 2, 3, 4, 5, or 6 amino acids are substituted; and at least one of the following substitutions as determined with reference to SEQ ID NO:2: T at position 20; K at position 39; I at position 48; or A, T, or Y at position 49; or
(b) the V_{H} region (i) comprises the amino acid sequence of SEQ ID NO:1; or (ii) has at least 70% identity to SEQ ID NO1: and comprises a CDR1 as set forth in Table 1 or the CDR1 of Table 1 in which 1, 2, or 3 amino acids are substituted, a CDR2 as set forth in Table 1 or the CDR2 of Table 1 in which 1, 2, 3, 4, 5, 6, 7, 8, or 9 amino acids are substituted, and a CDR3 as set forth in Table 1 or the CDR3 of Table 1 in which 1, 2, 3, 4, 5, 6, or 7 amino acids are substituted; and at least one of the following substitutions as determined with reference to SEQ ID NO:1: V at position 2, A or E at position 23, A at position 40, K at position 43, or E at position 46; and
the V_{L} region comprises an amino acid sequence having at least 70% identity to SEQ ID NO:2 and comprises a CDR1 as set forth in Table 1 or the CDR1 of Table 1 in which 1, 2, 3, or 4 amino acids are substituted, a CDR2 as set forth in Table 1 or the CDR2 of Table 1 in which 1, 2, or 3 amino acids are substituted, and a CDR3 as set forth in Table 1 or the CDR3 of Table 1 in which 1, 2, 3, 4, 5, or 6 amino acids are substituted; and at least one of the following substitutions as determined with reference to SEQ ID NO:2: T at position 20; K at position 39; I at position 48; or A, T, or Y at position 49.

10. (canceled)

11. The anti-CSP antibody of claim 9, wherein the V_{H} comprises an amino acid sequence having at least 80% identity to SEQ ID NO:1; and/or the V_{L} region comprises an amino acid sequence having at least 80% identity to SEQ ID NO:2.

12. The anti-CSP antibody of claim 9 wherein the V_{H} comprises an amino acid sequence having at least 90% identity to SEQ ID NO:1; and/or the V_{L} region comprises an amino acid sequence having at least 90% identity to SEQ ID NO:2.

13. An anti-CSP antibody comprising a V_{H} region and V_{L} region, wherein
the V_{H} region has at least 90% identity to any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95; and comprises at least one substitution as determined with reference to SEQ ID NO:1, wherein the at least one substitution is selected from the group consisting of V at position 2; A at position 23; S, D, or N at position 31; S at position 33; A at position 40; E at position 46; N or Q at position 53; D at position 54; E at position 55; R or N at position 56; Q at position 57; A at position 61; E at position 62; K at position 65; A at position 100; A, T, Q, or H at position 102; T at position 103; T at position 104; Y at position 105; and Y at position 108; and
the V_{L} region has at least 90% identity to any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94; or 96; and comprises at least one substitution as determined with reference to SEQ ID NO:2, wherein the at least one substitution is selected from the group consisting T at position 20; D at position 28; D at position 30, S at position 31; I at position 48; A or T at position 49; K, Q, S, L, V, or A at position 50; S or N at position 53, D at position 56; Y at position 92; H at position 94, and S or Y at position 95.

14. The anti-CSP antibody of claim 13, wherein the antibody comprises the following sets of amino acids:
(i) A at position 23 and A at position 40 as determined with reference to SEQ ID NO:1; and T at position 20 as determined with reference to SEQ ID NO:2;
(ii) E at position 46 and A at position 61 as determined with reference to SEQ ID NO:1;
(iii) V at position 2, A at position 23, and A at position 40 as determined with reference to SEQ ID NO: 1; and T at position 20 and I at position 48 as determined with reference to SEQ ID NO:2;
(iv) A at position 23, A at position 40, E at position 46, and A at position 61 as determined with reference to SEQ ID NO:1; and T at position 20 as determined with reference to SEQ ID NO:2;
(v) V at position 2, A at position 23, A at position 40, E at position 46, and A at position 61 as determined with reference to SEQ ID NO: 1; and T at position 20 and I at position 48 as determined with reference to SEQ ID NO:2;
(vi) N at position 53 and E at position 55 as determined with reference to SEQ ID NO:1; or
(vii) A at position 23 and A at position 40 as determined with reference to SEQ ID NO:1; and T at position 20 and Y at position 95 as determined with reference to SEQ ID NO:2.

15. The anti-CSP antibody of claim 13, where in the V_{H} region has at least 95% identity to any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95; and/or the V_{L} region has at least 95% identity to any one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96.

16. The anti-CSP antibody of claim 13, wherein the V_{H} region comprises the amino acid sequence of any one of SEQ ID NOS:3, 5, 7, 9, 11, 13, 15, 17, 19, 21, 23, 25, 27, 29, 31, 33, 35, 37, 39, 41, 43, 45, 47, 49, 51, 53, 55, 57, 79, 61, 63, 65, 67, 69, 71, 73, 75, 77, 79, 81, 83, 85, 87, 89, 91, 93, or 95; and/or the V_{L} region comprises the amino acid sequence of any one of SEQ ID NOS:4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, or 96.

17. The anti-CSP antibody of claim 13, wherein the antibody comprises:
a V_{H} region comprising amino acid sequence SEQ ID NO:3; and a V_{L} region comprising amino acid sequence SEQ ID NO:4;
a V_{H} region comprising amino acid sequence SEQ ID NO:5; and a V_{L} region comprising amino acid sequence SEQ ID NO:6;
a V_{H} region comprising amino acid sequence SEQ ID NO:7; and a V_{L} region comprising amino acid sequence SEQ ID NO:8;
a V_{H} region comprising amino acid sequence SEQ ID NO:9; and a V_{L} region comprising amino acid sequence SEQ ID NO:10;
a V_{H} region comprising amino acid sequence SEQ ID NO:11; and a V_{L} region comprising amino acid sequence SEQ ID NO:12;
a V_{H} region comprising amino acid sequence SEQ ID NO:13; and a V_{L} region comprising amino acid sequence SEQ ID NO:14;
a V_{H} region comprising amino acid sequence SEQ ID NO:15; and a V_{L} region comprising amino acid sequence SEQ ID NO:16;
a V_{H} region comprising amino acid sequence SEQ ID NO:17; and a V_{L} region comprising amino acid sequence SEQ ID NO:18;
a V_{H} region comprising amino acid sequence SEQ ID NO:19; and a V_{L} region comprising amino acid sequence SEQ ID NO:20;
a V_{H} region comprising amino acid sequence SEQ ID NO:21; and a V_{L} region comprising amino acid sequence SEQ ID NO:22;
a V_{H} region comprising amino acid sequence SEQ ID NO:23; and a V_{L} region comprising amino acid sequence SEQ ID NO:24;
a V_{H} region comprising amino acid sequence SEQ ID NO:25; and a V_{L} region comprising amino acid sequence SEQ ID NO:26;
a V_{H} region comprising amino acid sequence SEQ ID NO:27 and a V_{L} region comprising amino acid sequence SEQ ID NO:28;
a V_{H} region comprising amino acid sequence SEQ ID NO:29 and a V_{L} region comprising amino acid sequence SEQ ID NO:30;
a V_{H} region comprising amino acid sequence SEQ ID NO:31 and a V_{L} region comprising amino acid sequence SEQ ID NO:32;
a V_{H} region comprising amino acid sequence SEQ ID NO:33 and a V_{L} region comprising amino acid sequence SEQ ID NO:34;
a V_{H} region comprising amino acid sequence SEQ ID NO:35 and a V_{L} region comprising amino acid sequence SEQ ID NO:36;
a V_{H} region comprising amino acid sequence SEQ ID NO:27 and a V_{L} region comprising amino acid sequence SEQ ID NO:38;
a V_{H} region comprising amino acid sequence SEQ ID NO:39 and a V_{L} region comprising amino acid sequence SEQ ID NO:40;
a V_{H} region comprising amino acid sequence SEQ ID NO:41 and a V_{L} region comprising amino acid sequence SEQ ID NO:42;
a V_{H} region comprising amino acid sequence SEQ ID NO:43 and a V_{L} region comprising amino acid sequence SEQ ID NO:44;
a V_{H} region comprising amino acid sequence SEQ ID NO:45 and a V_{L} region comprising amino acid sequence SEQ ID NO:46;
a V_{H} region comprising amino acid sequence SEQ ID NO:47 and a V_{L} region comprising amino acid sequence SEQ ID NO:48;
a V_{H} region comprising amino acid sequence SEQ ID NO:49 and a V_{L} region comprising amino acid sequence SEQ ID NO:50;
a V_{H} region comprising amino acid sequence SEQ ID NO:51 and a V_{L} region comprising amino acid sequence SEQ ID NO:52;
a V_{H} region comprising amino acid sequence SEQ ID NO:53 and a V_{L} region comprising amino acid sequence SEQ ID NO:54;
a V_{H} region comprising amino acid sequence SEQ ID NO:55 and a V_{L} region comprising amino acid sequence SEQ ID NO:56;
a V_{H} region comprising amino acid sequence SEQ ID NO:57 and a V_{L} region comprising amino acid sequence SEQ ID NO:58;
a V_{H} region comprising amino acid sequence SEQ ID NO:59 and a V_{L} region comprising amino acid sequence SEQ ID NO:60;
a V_{H} region comprising amino acid sequence SEQ ID NO:61 and a V_{L} region comprising amino acid sequence SEQ ID NO:62;
a V_{H} region comprising amino acid sequence SEQ ID NO:63 and a V_{L} region comprising amino acid sequence SEQ ID NO:64;
a V_{H} region comprising amino acid sequence SEQ ID NO:65 and a V_{L} region comprising amino acid sequence SEQ ID NO:66;
a V_{H} region comprising amino acid sequence SEQ ID NO:67 and a V_{L} region comprising amino acid sequence SEQ ID NO:68;
a V_{H} region comprising amino acid sequence SEQ ID NO:69 and a V_{L} region comprising amino acid sequence SEQ ID NO:50;
a V_{H} region comprising amino acid sequence SEQ ID NO:71 and a V_{L} region comprising amino acid sequence SEQ ID NO:72;
a V_{H} region comprising amino acid sequence SEQ ID NO:73 and a V_{L} region comprising amino acid sequence SEQ ID NO:74;
a V_{H} region comprising amino acid sequence SEQ ID NO:75 and a V_{L} region comprising amino acid sequence SEQ ID NO:76;
a V_{H} region comprising amino acid sequence SEQ ID NO:77 and a V_{L} region comprising amino acid sequence SEQ ID NO:78;
a V_{H} region comprising amino acid sequence SEQ ID NO:79 and a V_{L} region comprising amino acid sequence SEQ ID NO:80;
a V_{H} region comprising amino acid sequence SEQ ID NO:81 and a V_{L} region comprising amino acid sequence SEQ ID NO:82;
a V_{H} region comprising amino acid sequence SEQ ID NO:83 and a V_{L} region comprising amino acid sequence SEQ ID NO:84;
a V_{H} region comprising amino acid sequence SEQ ID NO:85 and a V_{L} region comprising amino acid sequence SEQ ID NO:86;
a V_{H} region comprising amino acid sequence SEQ ID NO:87 and a V_{L} region comprising amino acid sequence SEQ ID NO:88;
a V_{H} region comprising amino acid sequence SEQ ID NO:89 and a V_{L} region comprising amino acid sequence SEQ ID NO:90;
a V_{H} region comprising amino acid sequence SEQ ID NO:91 and a V_{L} region comprising amino acid sequence SEQ ID NO:92;
a V_{H} region comprising amino acid sequence SEQ ID NO:93 and a V_{L} region comprising amino acid sequence SEQ ID NO:94; or
a V_{H} region comprising amino acid sequence SEQ ID NO:95 and a V_{L} region comprising amino acid sequence SEQ ID NO:96.

18. An expression vector comprising a polynucleotide encoding the V_{H} region and/or the V_{L} region of the anti-CSP antibody of claim 1.

19. A host cell that comprises an expression vector of claim 18.

20. A host cell comprising a polynucleotide that encodes the V_{H} region and/or the V_{L} region of the anti-CSP antibody of claim 1.

21. A method of producing the anti-CSP antibody of claim 1, the method comprising culturing the host cell of claim 20 in a culture medium.

22. A method of treating or preventing malaria, the method comprising administering the anti-CSP antibody of claim 1 to a patient that has malaria or is at risk of contracting malaria.

23.-24. (canceled)


SEQUENCE LISTING (122 sequences)

SEQ ID NO: 1 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSSFFDFWGQGTLVTVSS

SEQ ID NO: 2 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASVLESGVPSRFSGSGSGTEFTLTISSLQPDDFATYYCQHYNSYFVTFGQGTKVEIK

SEQ ID NO: 3 - protein, 119 aa, Artificial Sequence
  source: /note=“Description of Artificial Sequence Synthetic polypeptide”
QMQLVESGGGVVQPGRSLRLSCAASGFTFSTYAMHWVRQAPGQGLQWVAVISYHSTNKYYEDSVRGRFTISRDNSKNTLYLQMNSLRAEDTAVYYCARDGYSSSFFDFWGQGTLVTVSS

SEQ ID NO: 4 - protein, 107 aa, Artificial Sequence
  source: /note=“Description of Artificial Sequence Synthetic polypeptide”
DIQMTQSPSTLSASVGDRVTITCRASQSISRWLAWYQQQPGKAPKLLMSGASVLESGVPSRFSGSGSGTEFTLTISSLQPDDFATYYCQHYNSYFVTFGQGTKVEIK

SEQ ID NO: 5 - protein, 119 aa, Artificial Sequence
  source: /note=“Description of Artificial Sequence Synthetic polypeptide”
QVQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSSFFDFWGQGTLVTVSS

SEQ ID NO: 6 - protein, 107 aa, Artificial Sequence; identical to SEQ ID NO: 2
  source: /note=“Description of Artificial Sequence Synthetic polypeptide”

SEQ ID NO: 7 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLEWVAVISYHSTNKYYADSVRGRFTISRDNSKNTLYLQMNSLRAEDTAVYYCARDGYSSSFFDFWGQGTLVTVSS

SEQ ID NO: 8 - protein, 107 aa, Artificial Sequence; identical to SEQ ID NO: 2
  source: /note=“Description of Artificial Sequence Synthetic polypeptide”

SEQ ID NO: 9 - protein, 119 aa, Artificial Sequence; identical to SEQ ID NO: 1
  source: /note=“Description of Artificial Sequence Synthetic polypeptide”

SEQ ID NO: 10 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLISGASVLESGVPSRFSGSGSGTEFTLTISSLQPDDFATYYCQHYNSYFVTFGQGTKVEIK

SEQ ID NO: 11 - protein, 119 aa, Artificial Sequence
  source: /note=“Description of Artificial Sequence Synthetic polypeptide”
QVQLVESGGGVVQPGRSLRLSCAASGFTFSTYAMHWVRQAPGQGLQWVAVISYHSTNKYYEDSVRGRFTISRDNSKNTLYLQMNSLRAEDTAVYYCARDGYSSSFFDFWGQGTLVTVSS

SEQ ID NO: 12 - protein, 107 aa, Artificial Sequence
  source: /note=“Description of Artificial Sequence Synthetic polypeptide”
DIQMTQSPSTLSASVGDRVTITCRASQSISRWLAWYQQQPGKAPKLLISGASVLESGVPSRFSGSGSGTEFTLTISSLQPDDFATYYCQHYNSYFVTFGQGTKVEIK

SEQ ID NO: 13 - protein, 119 aa, Artificial Sequence
  source: /note=“Description of Artificial Sequence Synthetic polypeptide”
QMQLVESGGGVVQPGRSLRLSCAASGFTFSTYAMHWVRQAPGQGLEWVAVISYHSTNKYYADSVRGRFTISRDNSKNTLYLQMNSLRAEDTAVYYCARDGYSSSFFDFWGQGTLVTVSS

SEQ ID NO: 14 - protein, 107 aa, Artificial Sequence; identical to SEQ ID NO: 4
  source: /note=“Description of Artificial Sequence Synthetic polypeptide”

SEQ ID NO: 15 - protein, 119 aa, Artificial Sequence
  source: /note=“Description of Artificial Sequence Synthetic polypeptide”
QVQLVESGGGVVQPGRSLRLSCAASGFTFSTYAMHWVRQAPGQGLEWVAVISYHSTNKYYADSVRGRFTISRDNSKNTLYLQMNSLRAEDTAVYYCARDGYSSSFFDFWGQGTLVTVSS

SEQ ID NO: 16 - protein, 107 aa, Artificial Sequence; identical to SEQ ID NO: 12
  source: /note=“Description of Artificial Sequence Synthetic polypeptide”

SEQ ID NO: 17 - protein, 119 aa, Artificial Sequence
  source: /note=“Description of Artificial Sequence Synthetic polypeptide”
QMQLVESGGGVVQPGRSLRLSCTASGFTFSSYAMHWVRQSPGQGLQWVAVISYHSTNKYYEDSVRGRFTISRDNSKNTLYLQMNSLRAEDTAVYYCARDGYSSSFFDFWGQGTLVTVSS

SEQ ID NO: 18 - protein, 107 aa, Artificial Sequence; identical to SEQ ID NO: 2
  source: /note=“Description of Artificial Sequence Synthetic polypeptide”

SEQ ID NO: 19 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYDSTNKYYEDSVRGRFTISRDNSKNTLYLQMNSLRAEDTAVYYCARDGYSSSFFDFWGQGTLVTVSS

SEQ ID NO: 20 - protein, 107 aa, Artificial Sequence; identical to SEQ ID NO: 2
  source: /note=“Description of Artificial Sequence Synthetic polypeptide”

SEQ ID NO: 21 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVKGRFTISRDNSKNTLYLQMNSLRAEDTAVYYCARDGYSSSFFDFWGQGTLVTVSS

SEQ ID NO: 22 - protein, 107 aa, Artificial Sequence; identical to SEQ ID NO: 2
  source: /note=“Description of Artificial Sequence Synthetic polypeptide”

SEQ ID NO: 23 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSSFFDYWGQGTLVTVSS

SEQ ID NO: 24 - protein, 107 aa, Artificial Sequence; identical to SEQ ID NO: 2
  source: /note=“Description of Artificial Sequence Synthetic polypeptide”

SEQ ID NO: 25 - protein, 119 aa, Artificial Sequence; identical to SEQ ID NO: 1
  source: /note=“Description of Artificial Sequence Synthetic polypeptide”

SEQ ID NO: 26 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASVLESGVPSRFSGSGSGTEFTLTISSLQPDDFATYYCQHYNSYSVTFGQGTKVEIK

SEQ ID NO: 27 - protein, 119 aa, Artificial Sequence
  source: /note=“Description of Artificial Sequence Synthetic polypeptide”
QMQLVESGGGVVQPGRSLRLSCTASGFTFSDYAMHWVRQSPGQGLQWVAVISYHSTNKYYEDSVRGRFTISRDNSKNTLYLQMNSLRAEDTAVYYCARDGYSSSFFDFWGQGTLVTVSS

SEQ ID NO: 28 - protein, 107 aa, Artificial Sequence; identical to SEQ ID NO: 2
  source: /note=“Description of Artificial Sequence Synthetic polypeptide”

SEQ ID NO: 29 - protein, 119 aa, Artificial Sequence; identical to SEQ ID NO: 1
  source: /note=“Description of Artificial Sequence Synthetic polypeptide”

SEQ ID NO: 30 - protein, 107 aa, Artificial Sequence
  source: /note=“Description of Artificial Sequence Synthetic polypeptide”
DIQMTQSPSTLSASVGDRVAITCRASQDISRWLAWYQQQPGKAPKLLMSGASVLESGVPSRFSGSGSGTEFTLTISSLQPDDFATYYCQHYNSYFVTFGQGTKVEIK

SEQ ID NO: 31 - protein, 119 aa, Artificial Sequence; identical to SEQ ID NO: 1
  source: /note=“Description of Artificial Sequence Synthetic polypeptide”

SEQ ID NO: 32 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASVLEDGVPSRFSGSGSGTEFTLTISSLQPDDFATYYCQHYNSYFVTFGQGTKVEIK

SEQ ID NO: 33 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEESVRGRFTISRDNSKNTLYLQMNSLRAEDTAVYYCARDGYSSSFFDFWGQGTLVTVSS

SEQ ID NO: 34 - protein, 107 aa, Artificial Sequence; identical to SEQ ID NO: 2
  source: /note=“Description of Artificial Sequence Synthetic polypeptide”

SEQ ID NO: 35 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDAYSSSFFDFWGQGTLVTVSS

SEQ ID NO: 36 - protein, 107 aa, Artificial Sequence; identical to SEQ ID NO: 2
  source: /note=“Description of Artificial Sequence Synthetic polypeptide”

SEQ ID NO: 37 - protein, 119 aa, Artificial Sequence; identical to SEQ ID NO: 1
  source: /note=“Description of Artificial Sequence Synthetic polypeptide”

SEQ ID NO: 38 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASVLESGVPSRFSGSGSGTEFTLTISSLQPDDFATYYCQHYYSYFVTFGQGTKVEIK

SEQ ID NO: 39 - protein, 119 aa, Artificial Sequence
  source: /note=“Description of Artificial Sequence Synthetic polypeptide”
QMQLVESGGGVVQPGRSLRLSCTASGFTFSNYAMHWVRQSPGQGLQWVAVISYHSTNKYYEDSVRGRFTISRDNSKNTLYLQMNSLRAEDTAVYYCARDGYSSSFFDFWGQGTLVTVSS

SEQ ID NO: 40 - protein, 107 aa, Artificial Sequence; identical to SEQ ID NO: 2
  source: /note=“Description of Artificial Sequence Synthetic polypeptide”

SEQ ID NO: 41 - protein, 119 aa, Artificial Sequence
  source: /note=“Description of Artificial Sequence Synthetic polypeptide”
QMQLVESGGGVVQPGRSLRLSCTASGFTFSTYSMHWVRQSPGQGLQWVAVISYHSTNKYYEDSVRGRFTISRDNSKNTLYLQMNSLRAEDTAVYYCARDGYSSSFFDFWGQGTLVTVSS

SEQ ID NO: 42 - protein, 107 aa, Artificial Sequence; identical to SEQ ID NO: 2
  source: /note=“Description of Artificial Sequence Synthetic polypeptide”

SEQ ID NO: 43 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISNHETNKYYEDSVRGRFTISRDNSKNTLYLQMNSLRAEDTAVYYCARDGYSSSFFDFWGQGTLVTVSS

SEQ ID NO: 44 - protein, 107 aa, Artificial Sequence; identical to SEQ ID NO: 2
  source: /note=“Description of Artificial Sequence Synthetic polypeptide”

SEQ ID NO: 45 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISQHSTNKYYEDSVRGRFTISRDNSKNTLYLQMNSLRAEDTAVYYCARDGYSSSFFDFWGQGTLVTVSS

SEQ ID NO: 46 - protein, 107 aa, Artificial Sequence; identical to SEQ ID NO: 2
  source: /note=“Description of Artificial Sequence Synthetic polypeptide”

SEQ ID NO: 47 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSRNKYYEDSVRGRFTISRDNSKNTLYLQMNSLRAEDTAVYYCARDGYSSSFFDFWGQGTLVTVSS

SEQ ID NO: 48 - protein, 107 aa, Artificial Sequence; identical to SEQ ID NO: 2
  source: /note=“Description of Artificial Sequence Synthetic polypeptide”

SEQ ID NO: 49 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSNNKYYEDSVRGRFTISRDNSKNTLYLQMNSLRAEDTAVYYCARDGYSSSFFDFWGQGTLVTVSS

SEQ ID NO: 50 - protein, 107 aa, Artificial Sequence; identical to SEQ ID NO: 2
  source: /note=“Description of Artificial Sequence Synthetic polypeptide”

SEQ ID NO: 51 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTQKYYEDSVRGRFTISRDNSKNTLYLQMNSLRAEDTAVYYCARDGYSSSFFDFWGQGTLVTVSS

SEQ ID NO: 52 - protein, 107 aa, Artificial Sequence; identical to SEQ ID NO: 2
  source: /note=“Description of Artificial Sequence Synthetic polypeptide”

SEQ ID NO: 53 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYASSFFDFWGQGTLVTVSS

SEQ ID NO: 54 - protein, 107 aa, Artificial Sequence; identical to SEQ ID NO: 2
  source: /note=“Description of Artificial Sequence Synthetic polypeptide”

SEQ ID NO: 55 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYTSSFFDFWGQGTLVTVSS

SEQ ID NO: 56 - protein, 107 aa, Artificial Sequence; identical to SEQ ID NO: 2
  source: /note=“Description of Artificial Sequence Synthetic polypeptide”

SEQ ID NO: 57 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYQSSFFDFWGQGTLVTVSS

SEQ ID NO: 58 - protein, 107 aa, Artificial Sequence; identical to SEQ ID NO: 2
  source: /note=“Description of Artificial Sequence Synthetic polypeptide”

SEQ ID NO: 59 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYHSSFFDFWGQGTLVTVSS

SEQ ID NO: 60 - protein, 107 aa, Artificial Sequence; identical to SEQ ID NO: 2
  source: /note=“Description of Artificial Sequence Synthetic polypeptide”

SEQ ID NO: 61 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSTSFFDFWGQGTLVTVSS

SEQ ID NO: 62 - protein, 107 aa, Artificial Sequence; identical to SEQ ID NO: 2
  source: /note=“Description of Artificial Sequence Synthetic polypeptide”

SEQ ID NO: 63 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSTFFDFWGQGTLVTVSS

SEQ ID NO: 64 - protein, 107 aa, Artificial Sequence; identical to SEQ ID NO: 2
  source: /note=“Description of Artificial Sequence Synthetic polypeptide”

SEQ ID NO: 65 - protein, 119 aa, Artificial Sequence
  source: /note=“Description of Artificial Sequence Synthetic polypeptide”
QMQLVESGGGVVQPGRSLRLSCTASGFTFSTYAMHWVRQSPGQGLQWVAVISYHSTNKYYEDSVRGRFTISRDNSKNTLYLQMNSLRAEDTAVYYCARDGYSSSYFDFWGQGTLVTVSS

SEQ ID NO: 66 - protein, 107 aa, Artificial Sequence; identical to SEQ ID NO: 2
  source: /note=“Description of Artificial Sequence Synthetic polypeptide”

SEQ ID NO: 67 - protein, 119 aa, Artificial Sequence; identical to SEQ ID NO: 1
  source: /note=“Description of Artificial Sequence Synthetic polypeptide”

SEQ ID NO: 68 - protein, 107 aa, Artificial Sequence
  source: /note=“Description of Artificial Sequence Synthetic polypeptide”
DIQMTQSPSTLSASVGDRVAITCRASQSIDRWLAWYQQQPGKAPKLLMSGASVLESGVPSRFSGSGSGTEFTLTISSLQPDDFATYYCQHYNSYFVTFGQGTKVEIK

SEQ ID NO: 69 - protein, 119 aa, Artificial Sequence; identical to SEQ ID NO: 1
  source: /note=“Description of Artificial Sequence Synthetic polypeptide”

SEQ ID NO: 70 - protein, 107 aa, Artificial Sequence
  source: /note=“Description of Artificial Sequence Synthetic polypeptide”
DIQMTQSPSTLSASVGDRVAITCRASQSISSWLAWYQQQPGKAPKLLMSGASVLESGVPSRFSGSGSGTEFTLTISSLQPDDFATYYCQHYNSYFVTFGQGTKVEIK

SEQ ID NO: 71 - protein, 119 aa, Artificial Sequence; identical to SEQ ID NO: 1
  source: /note=“Description of Artificial Sequence Synthetic polypeptide”

SEQ ID NO: 72 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMAGASVLESGVPSRFSGSGSGTEFTLTISSLQPDDFATYYCQHYNSYFVTFGQGTKVEIK

SEQ ID NO: 73 - protein, 119 aa, Artificial Sequence; identical to SEQ ID NO: 1
  source: /note=“Description of Artificial Sequence Synthetic polypeptide”

SEQ ID NO: 74 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMTGASVLESGVPSRFSGSGSGTEFTLTISSLQPDDFATYYCQHYNSYFVTFGQGTKVEIK

SEQ ID NO: 75 - protein, 119 aa, Artificial Sequence; identical to SEQ ID NO: 1
  source: /note=“Description of Artificial Sequence Synthetic polypeptide”

SEQ ID NO: 76 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSKASVLESGVPSRFSGSGSGTEFTLTISSLQPDDFATYYCQHYNSYFVTFGQGTKVEIK

SEQ ID NO: 77 - protein, 119 aa, Artificial Sequence; identical to SEQ ID NO: 1
  source: /note=“Description of Artificial Sequence Synthetic polypeptide”

SEQ ID NO: 78 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSQASVLESGVPSRFSGSGSGTEFTLTISSLQPDDFATYYCQHYNSYFVTFGQGTKVEIK

SEQ ID NO: 79 - protein, 119 aa, Artificial Sequence; identical to SEQ ID NO: 1
  source: /note=“Description of Artificial Sequence Synthetic polypeptide”

SEQ ID NO: 80 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSSASVLESGVPSRFSGSGSGTEFTLTISSLQPDDFATYYCQHYNSYFVTFGQGTKVEIK

SEQ ID NO: 81 - protein, 119 aa, Artificial Sequence; identical to SEQ ID NO: 1
  source: /note=“Description of Artificial Sequence Synthetic polypeptide”

SEQ ID NO: 82 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSLASVLESGVPSRFSGSGSGTEFTLTISSLQPDDFATYYCQHYNSYFVTFGQGTKVEIK

SEQ ID NO: 83 - protein, 119 aa, Artificial Sequence; identical to SEQ ID NO: 1
  source: /note=“Description of Artificial Sequence Synthetic polypeptide”

SEQ ID NO: 84 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSVASVLESGVPSRFSGSGSGTEFTLTISSLQPDDFATYYCQHYNSYFVTFGQGTKVEIK

SEQ ID NO: 85 - protein, 119 aa, Artificial Sequence; identical to SEQ ID NO: 1
  source: /note=“Description of Artificial Sequence Synthetic polypeptide”

SEQ ID NO: 86 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSAASVLESGVPSRFSGSGSGTEFTLTISSLQPDDFATYYCQHYNSYFVTFGQGTKVEIK

SEQ ID NO: 87 - protein, 119 aa, Artificial Sequence; identical to SEQ ID NO: 1
  source: /note=“Description of Artificial Sequence Synthetic polypeptide”

SEQ ID NO: 88 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASSLESGVPSRFSGSGSGTEFTLTISSLQPDDFATYYCQHYNSYFVTFGQGTKVEIK

SEQ ID NO: 89 - protein, 119 aa, Artificial Sequence; identical to SEQ ID NO: 1
  source: /note=“Description of Artificial Sequence Synthetic polypeptide”

SEQ ID NO: 90 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASNLESGVPSRFSGSGSGTEFTLTISSLQPDDFATYYCQHYNSYFVTFGQGTKVEIK

SEQ ID NO: 91 - protein, 119 aa, Artificial Sequence; identical to SEQ ID NO: 1
  source: /note=“Description of Artificial Sequence Synthetic polypeptide”

SEQ ID NO: 92 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASVLESGVPSRFSGSGSGTEFTLTISSLQPDDFATYYCQHYNSHFVTFGQGTKVEIK

SEQ ID NO: 93 - protein, 119 aa, Artificial Sequence; identical to SEQ ID NO: 3
  source: /note=“Description of Artificial Sequence Synthetic polypeptide”

SEQ ID NO: 94 - protein, 107 aa, Artificial Sequence
  source: /note=“Description of Artificial Sequence Synthetic polypeptide”
DIQMTQSPSTLSASVGDRVTITCRASQSISRWLAWYQQQPGKAPKLLMSGASVLESGVPSRFSGSGSGTEFTLTISSLQPDDFATYYCQHYNSYYVTFGQGTKVEIK

SEQ ID NO: 95 - protein, 119 aa, Artificial Sequence; identical to SEQ ID NO: 1
  source: /note=“Description of Artificial Sequence Synthetic polypeptide”

SEQ ID NO: 96 - protein, 107 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASVLESGVPSRFSGSGSGTEFTLTISSLQPDDFATYYCQHYNSYYVTFGQGTKVEIK

SEQ ID NO: 97 - protein, 10 aa, Artificial Sequence
  source: /note=“Description of Artificial Sequence Synthetic peptide”
GFTFSTYAMH

SEQ ID NO: 98 - protein, 17 aa, Artificial Sequence
  source: /note=“Description of Artificial Sequence Synthetic peptide”
VISYHSTNKYYEDSVRG

SEQ ID NO: 99 - protein, 12 aa, Artificial Sequence
  source: /note=“Description of Artificial Sequence Synthetic peptide”
ARDGYSSSFFDF

SEQ ID NO: 100 - protein, 11 aa, Artificial Sequence
  source: /note=“Description of Artificial Sequence Synthetic peptide”
RASQSISRWLA

SEQ ID NO: 101 - protein, 7 aa, Artificial Sequence
  source: /note=“Description of Artificial Sequence Synthetic peptide”
GASVLES

SEQ ID NO: 102 - protein, 9 aa, Artificial Sequence
  source: /note=“Description of Artificial Sequence Synthetic peptide”
QHYNSYFVT

SEQ ID NO: 103 - protein, 24 aa, Plasmodium falciparum
NANPNANPNANPNANPNANPNANP

SEQ ID NO: 104 - protein, 12 aa, Plasmodium falciparum
NPNANPNANPNA

SEQ ID NO: 105 - protein, 20 aa, Plasmodium falciparum
NVDPNANPNVDPNANPNVDP

SEQ ID NO: 106 - protein, 16 aa, Plasmodium falciparum
NPDPNANPNVDPNANP

SEQ ID NO: 107 - protein, 12 aa, Plasmodium falciparum
DPNANPNVDPNA

SEQ ID NO: 108 - protein, 15 aa, Plasmodium falciparum
KQPADGNPDPNANPN

SEQ ID NO: 109 - protein, 8 aa, Artificial Sequence
  source: /note=“Description of Artificial Sequence Synthetic peptide”
GFTFSTYA

SEQ ID NO: 110 - protein, 8 aa, Artificial Sequence
  source: /note=“Description of Artificial Sequence Synthetic peptide”
ISYHSTNK

SEQ ID NO: 111 - protein, 6 aa, Artificial Sequence
  source: /note=“Description of Artificial Sequence Synthetic peptide”
QSISRW

SEQ ID NO: 112 - protein, 5 aa, Artificial Sequence
  source: /note=“Description of Artificial Sequence Synthetic peptide”
TYAMH

SEQ ID NO: 113 - protein, 10 aa, Artificial Sequence
  source: /note=“Description of Artificial Sequence Synthetic peptide”
DGYSSSFFDF

SEQ ID NO: 114 - protein, 7 aa, Artificial Sequence
  source: /note=“Description of Artificial Sequence Synthetic peptide”
GFTFSTY

SEQ ID NO: 115 - protein, 6 aa, Artificial Sequence
  source: /note=“Description of Artificial Sequence Synthetic peptide”
SYHSTN

SEQ ID NO: 116 - protein, 118 aa, Artificial Sequence
  source: /note=“Description of Artificial Sequence Synthetic polypeptide”
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGYSSSYFDYWGQGTLVTVSS

SEQ ID NO: 117 - protein, 106 aa, Artificial Sequence
  source: /note=“Description of Artificial Sequence Synthetic polypeptide”
DIQMTQSPSTLSASVGDRVAITCRASQSISRWLAWYQQQPGKAPKLLMSGASVLESGVPSRFSGSGSGTETLTISSLQPDDFATYYCQHYNSYFVTFGQGTKVEIK

SEQ ID NO: 118 - protein, 106 aa, Artificial Sequence
  source: /note=“Description of Artificial Sequence Synthetic polypeptide”
DIQMTQSPSTLSASVGDRVTITCRASQSISSWLAWYQQKPGKAPKLLIYKASSLESGVPSRFSGSGSGTETLTISSLQPDDFATYYCQQYNSYSWTFGQGTKVEIK

SEQ ID NO: 119 - protein, 12 aa, Plasmodium falciparum
NANPNANPNANP

SEQ ID NO: 120 - protein, 8 aa, Plasmodium falciparum
NANPNANP

SEQ ID NO: 121 - protein, 8 aa, Plasmodium falciparum
NVDPNANP

SEQ ID NO: 122 - protein, 4 aa, Plasmodium falciparum
NANP